(19) United States
(12) Patent Application Publication
(10) Pub. No.: US 2025/0235671 A1
(43) Pub. Date: Jul. 24, 2025

(54) FIXING STRUCTURE

(71) Applicant: HI-LEX CORPORATION, Hyogo (JP)

(72) Inventor: Shinsaku KOARASHI, Hyogo (JP)

(21) Appl. No.: 18/855,518

(22) PCT Filed: Apr. 28, 2023
(86) PCT No.: PCT/JP2023/016792

§ 371 Date: Oct. 9, 2024

(30) Foreign Application Priority Data
Apr. 28, 2022 (JP) 2022-075085

(51) Int. Cl. A61M 25/02

(52) U.S. Cl. CPC A61M 25/02; A61M 2025/024; A61M 2025/028

(57) ABSTRACT

The fixing structure of the present invention is a structure, comprising: a porous sheet composed of a non-resorbable material having biocompatibility; a cylindrical insertion part for guiding an insertion member from inside of a living body to outside of the living body; a sandwiching part for sandwiching a peripheral portion around the opening of the sheet in a thickness direction of the sheet and being fixed to the insertion part; and a fixing filamentary body composed of a filamentary body sewn to the sheet so as to fix the sheet and the sandwiching part.

### TECHNICAL FIELD

The present invention relates to a fixing structure.

### BACKGROUND ART

When an insertion member such as a medical tube and a driveline is led from inside of a living body to outside of the living body, a skin button, a cuff member, or the like is used as a fixture for fixing the insertion member to the skin.

As such a fixture, for example, Patent Document 1 discloses a medical tube fixture having a cylindrical part into which a medical tube is inserted, a flange part fixed at a percutaneous part, and a fixing nut for fixing the flange part to the cylindrical part.

### PRIOR ART DOCUMENT

### Patent Document

Patent Document 1: JP 2019-166310 A

### SUMMARY OF THE INVENTION

### Problems to be Solved by the Invention

Such a fixture as disclosed in Patent Document 1 fixes a flange part to a cylindrical part by sandwiching the flange part between a flange presser and a fixing nut. If the flange part is composed of a material having flexibility, like PTFE, the flange part deforms due to fastening of the fixing nut, generating a gap between the flange presser and the fixing nut. Therefore, treatment to prevent bacteria from entering through the gap is needed, which makes handling difficult.

Moreover, the flange part is fixed to the skin by a tissue penetrating into the flange part, but it takes time for the flange part to be completely fixed to the skin. Therefore, if the flange part moves with respect to the skin in an unfixed state before it is completely fixed to the skin, healing can be delayed.

Here, it is an object of the present invention to provide a fixing structure that can suppress intrusion of bacteria and suppress misalignment from a target site.

### Means to Solve the Problem

The fixing structure of the present invention comprises: a porous sheet having an opening through which an insertion member led from inside of a living body to outside of the living body is insertable, wherein the sheet is composed of a non-resorbable material having biocompatibility; a cylindrical insertion part having an insertion passage for guiding the insertion member from inside of the living body to outside of the living body; a sandwiching part for sandwiching a peripheral portion around the opening of the sheet in a thickness direction of the sheet and being fixed to the insertion part; and a fixing filamentary body composed of a filamentary body sewn to the sheet so as to fix the sheet and the sandwiching part, wherein the fixing filamentary body is sewn to a surface of the sheet so as to form a protruded and recessed structure providing resistance to a relative movement in an extending direction of the sheet with respect to a target site of the living body where the insertion member is inserted.

### Effects of the Invention

According to the fixing structure of the present invention, intrusion of bacteria can be suppressed, and misalignment from a target site can be suppressed, allowing for early fixation to the target site.

### BRIEF DESCRIPTION OF THE DRAWINGS

FIG. 1 is a cross-sectional view showing a state where a fixing structure of one embodiment of the present invention is embedded into a wall-like tissue.

FIG. 2 is a view showing one surface of the fixing structure of one embodiment of the present invention.

FIG. 3 is a view showing the other surface of the fixing structure of one embodiment of the present invention.

FIG. 4 is a schematic view of a cross section taken along a line IV-IV in FIG. 2.

FIG. 5 is an enlarged schematic view of a region A1 in FIG. 4.

FIG. 6 is an enlarged schematic view of a region A2 in FIG. 4.

FIG. 7 is an image of a cross section of the fixing structure of the present embodiment that has been removed together with the skin after a predetermined period of time has passed since the fixing structure was embedded.

FIG. 8 is an image of a cross section of the fixing structure of Comparative example that has been removed together with the skin after a predetermined period of time has passed since the fixing structure was embedded.

FIG. 9 is a schematic view showing a sewing pattern of a fixing structure of a second embodiment.

FIG. 10 is a schematic view showing a sewing pattern of a fixing structure of a third embodiment.

FIG. 11 is a schematic view showing a sewing pattern of a fixing structure of a fourth embodiment.

FIG. 12 is a schematic view showing a sewing pattern of a fixing structure of a fifth embodiment.

FIG. 13 is a perspective view showing a fixing structure of a sixth embodiment before a filamentary body is sewn.

FIG. 14 is a cross-sectional view taken along a line XIV-XIV in FIG. 13.

FIG. 15 is an exploded view showing a simplified fixing structure of the sixth embodiment.

FIG. 16 is a schematic view showing a state where respective members of the fixing structure are assembled to each other and the filamentary body is sewn from the state shown in FIG. 15.

FIG. 17 is an enlarged view of a part of the fixing structure in FIG. 16 where the filamentary body is sewn.

FIG. 18 is a view showing a state where a sandwiching part is mounted to a sheet in the sixth embodiment.

FIG. 19 is a plan view showing a first sandwiching member of the sandwiching part in the sixth embodiment.

FIG. 20 is a plan view of a sheet in the sixth embodiment.

FIG. 21 is a photograph showing a state where a sheet-side filamentary body is sewn to the sheet in the sixth embodiment.

FIG. 22 is a plan view showing a second sandwiching member of the sandwiching part in the sixth embodiment.

FIG. 23 is a schematic view showing a simplified one example of a sewing pattern in which a fixing filamentary body and a sheet-side filamentary body are sewn.

FIG. 24 is a schematic view showing a sewing pattern of a sheet-side filamentary body in a fixing structure of a seventh embodiment.

FIG. 25 is a schematic view showing a variation of the fixing structure of the seventh embodiment.

FIG. 26 is a schematic view showing a sewing pattern of a sheet-side filamentary body in a fixing structure of an eighth embodiment.

FIG. 27 is a view schematically showing an overlap between the sheet-side filamentary body and a fixing filamentary body in the fixing structure of the eighth embodiment.

FIG. 28 is a schematic view showing a sewing pattern of a sheet-side filamentary body in a fixing structure of a ninth embodiment.

### EMBODIMENT FOR CARRYING OUT THE INVENTION

### First Embodiment

The fixing structure of one embodiment of the present invention will be described below with reference to the drawings. It should be noted that embodiments shown below are merely examples, and the fixing structure of the present invention is not limited to the following embodiments.

It should be noted that, in the present specification, “perpendicular to A” and similar expressions do not only refer to a direction strictly perpendicular to A, but also refer to the direction including being substantially perpendicular to A. Moreover, in the present specification, “parallel to B” and similar expressions do not only refer to a direction strictly parallel to B, but also refer to the direction including being substantially parallel to B. In addition, in the present specification, “C-shape” and similar expressions do not only refer to a strict C-shape, but also refer to the shape including a shape visually associated with a C-shape (substantially a C-shape).

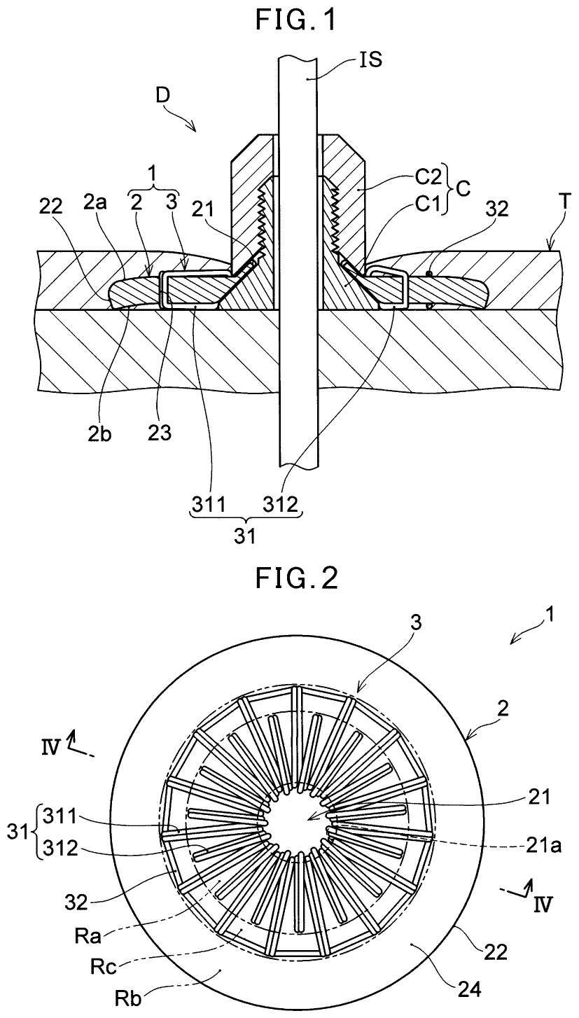

As shown in FIG. 1, the fixing structure 1 of this embodiment is used when fixing an insertion member IS, which is led out from inside of a living body to outside of the living body, to a wall-like tissue T of the living body. As shown in FIG. 1, the fixing structure 1 is fixed to the wall-like tissue T of the living body by being embedded at a predetermined position into a predetermined depth in the wall-like tissue T with the insertion member IS inserted through the fixing structure 1. Applications of the fixing structure 1 are not particularly limited as long as they are for fixing the insertion member IS, which is led from inside of the living body to outside of the living body, to the wall-like tissue T of the living body. For example, the fixing structure 1 can be used for a skin button (cuff member), etc. into which the insertion member IS is inserted.

In the present specification, the term “living body” refers to a body of a human or a non-human animal. The term “wall-like tissue” refers to any tissue such as a wall and a membrane of a living body having a predetermined thickness, into which an insertion member IS is inserted. Specifically, the wall-like tissue refers to such as skins (epidermis, dermis, subcutaneous tissue), a muscle layer, and various tissues inside of the living body that constitute various organs, as well as layers formed by combining them. In this embodiment, the wall-like tissue T into which the insertion member IS is inserted is a tissue including skins (epidermis, dermis, subcutaneous tissue) and a muscle layer. A fixing position (embedding position) in the wall-like tissue T to which the fixing structure 1 is fixed can be changed as appropriate depending on a body type, etc. of a patient (living body) to which the fixing structure 1 is fixed.

Applications of the fixing structure 1 are not particularly limited as long as they are for fixing the insertion member IS, which is led from inside of the living body to outside of the living body, to the wall-like tissue T of the living body. In this embodiment, the fixing structure 1 is used when inserting into an abdominal wall-like tissue T a drive line (insertion member IS) used in a medical instrument (e.g., an artificial organ such as a ventricular assist device (VAD) and an artificial lung) implanted into a human body and leading the drive line from inside of the living body to outside of the living body of the human body. The drive line led out to outside of the living body is fixed at a predetermined position in the abdomen via the fixing structure 1 as shown in FIG. 1.

The insertion member IS is a linear member having a predetermined length that is inserted into a wall-like tissue T of a living body. It should be noted that the term “linear” for an insertion member IS means that the insertion member IS extends a predetermined length, regardless of whether the insertion member IS is hollow or solid. In this embodiment, the insertion member IS is a long member for medical use that is placed in a state where it penetrates through the wall-like tissue T of the living body. More specifically, the insertion member IS is a drive line for an artificial organ (ventricular assist device). One end of the insertion member IS is coupled to an artificial organ (not shown) placed at a predetermined position inside of the living body, and the other end is coupled to an equipment (such as a power source) placed outside of the living body.

An internal structure of the insertion member IS is not particularly limited, but, for example, the insertion member IS has a cooling water circulating passage for circulating a cooling water between an artificial organ inside of the living body and a pressure pump outside of the living body and a power cable for coupling the artificial organ inside of the living body to a power source outside of the living body. It should be noted that the insertion member IS may be configured to have only a power cable or have only a cooling water circulating passage. Moreover, the insertion member IS may be configured to have a member having functions other than those described above or may be configured as a hollow member (tube) having no members inside. When the insertion member IS is used as a hollow member that communicates between inside of the living body and outside of the living body, it may be used to deliver a drug for treatment on a treatment site from outside of the living body to inside of the living body.

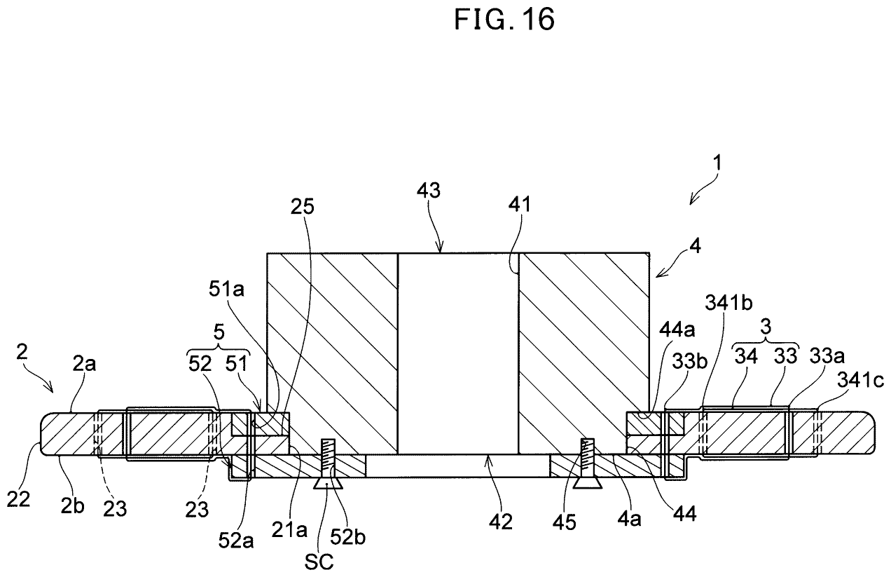

When the insertion member IS is inserted into the wall-like tissue T, the fixing structure 1 is fixed to the wall-like tissue T with the insertion member IS inserted into the fixing structure 1, as shown in, for example, FIG. 1. More specifically, a fixing device D shown in FIG. 1 is used to fix the insertion member IS to the wall-like tissue T with the insertion member IS inserted into the wall-like tissue T. The fixing structure 1 (fixing device D) of this embodiment comprises a connecting member C that (indirectly) connects the insertion member IS to a sheet 2. In this embodiment, the connecting member C comprises a first connecting member C1 and a second connecting member C2 that sandwich a sheet 2, which will be mentioned later, of the fixing structure 1 in a thickness direction of the sheet 2. The first connecting member C1 and the second connecting member C2 are configured so that the insertion member IS is insertable therethrough. In this embodiment, at least one of the first connecting member C1 and the second connecting member C2 (the first connecting member C1 in this embodiment) is inserted through an opening 21 of the sheet 2. The first connecting member C1 and the second connecting member C2 through which the insertion member IS is inserted are screwed each other, so that a peripheral portion around the opening 21 of the sheet 2 is sandwiched between the first connecting member C1 and the second connecting member C2. The fixing structure 1 and the insertion member IS are thereby connected via a connecting member C. In this state, as shown in FIG. 1, the fixing structure 1 is embedded into the wall-like tissue T, so that the insertion member IS is fixed at a predetermined position via the fixing structure 1 with the insertion member IS inserted into the wall-like tissue T. It should be noted that a method of connecting the connecting member C and the sheet 2 is not limited to the above-mentioned connecting method. Moreover, a structure of the fixing structure 1 is not limited to the structure using the connecting member C shown in FIG. 2 as long as it can support the insertion member IS with being inserted. For example, as shown in FIG. 16, etc., the fixing structure 1 may comprise a cylindrical insertion part 4 and a sandwiching part 5, and the insertion part 4 and the sandwiching part 5 may be fixed with each other by a fixing filamentary body 33.

As shown in FIGS. 1 to 3, the fixing structure 1 comprises a porous sheet 2 having an opening 21 through which an insertion member IS led from inside of a living body to outside of the living body is insertable, and the sheet 2 is composed of a non-resorbable material having biocompatibility.

The sheet 2 is a part to be embedded into the wall-like tissue T when the fixing structure 1 is fixed (anchored) to the wall-like tissue T with the insertion member IS inserted into the wall-like tissue T. For example, the sheet 2 is embedded into the wall-like tissue T so as to be slipped under a part of the wall-like tissue T (e.g. skin) incised for the insertion member IS to be inserted through. The sheet 2 is thereby fixed to a predetermined site of the wall-like tissue T, as shown in FIG. 1.

The sheet 2 is composed of a non-resorbable material having biocompatibility. The non-resorbable material having biocompatibility that constitutes the sheet 2 can be a similar material as that used inside of a body or at a wound site as a medical sheet. Moreover, the sheet 2 is preferably composed of a material having flexibility in a state before the filamentary body 3, which will be mentioned later, is sewn thereto. The material constituting the sheet 2 can be, for example, at least one selected from the group consisting of a polyurethane resin, a polyamide resin, a polylactic acid resin, a polyolefin resin, a polyester resin, a fluororesin, a urea resin, a phenolic resin, an epoxy resin, a polyimide resin, an acrylic resin, a methacrylic resin, and derivatives thereof, and it can be preferably an expanded polytetrafluoroethylene (ePTFE).

Moreover, in order to promote growth and anchoring of a cell of the wall-like tissue T into the sheet 2, the sheet 2 is preferably composed of a porous sheet. A pore size (an average pore size) of the porous sheet 2 is not particularly limited as long as it is a size to allow cells to easily grow and anchor when the sheet 2 is embedded into the wall-like tissue T. The average pore size of the sheet 2 can be, for example, 10 to 100 μm.

A shape of the sheet 2 is not particularly limited as long as it is a shape that allows the insertion member IS to be inserted through the sheet 2 and is suitable for embedding into wall-like tissue T. In this embodiment, the sheet 2 is formed in a circular (substantially circular) shape having an opening 21 in the central portion, but may be formed in a shape other than the circular shape, such as an elliptical shape and a polygonal shape. A size of the sheet 2 is not particularly limited, and is changed as appropriate depending on a diameter of an insertion member IS to be inserted into the sheet 2, an application of the fixing structure 1, or a fixing position. For example, the sheet 2 can have a size similar to that of a flange part of a known skin button (cuff member) to be embedded into the wall-like tissue T. When the sheet 2 is circular, the diameter of the sheet 2 can be, for example, 5 to 80 mm. Moreover, a thickness of the sheet 2 is not particularly limited, but can be, for example, 0.5 to 5 mm. Furthermore, when the sheet 2 has a longitudinal dimension different from a lateral dimension, such as an elliptical shape, the longitudinal dimension of the sheet 2 can be 10 to 80 mm. For example, when the sheet 2 has an elliptical shape, the major axis of the sheet 2 can be 40 to 80 mm, and the minor axis can be 20 to 40 mm.

The opening 21 provided in the sheet 2 penetrates the central portion of the sheet 2 in the thickness direction of the sheet 2. A size of the opening 21 is appropriately set so that the insertion member IS is insertable through the opening 21. In this embodiment, the opening 21 is provided with a size that allows the connection member C (see FIG. 1) to be inserted through the opening 21 (with a filamentary body, which will be mentioned later, provided). It should be noted that in the present specification, a predetermined region in a direction from an opening edge 21a of the opening 21 toward an outer peripheral edge 22 of the sheet 2 (an outer side in a radius direction of the sheet 2, in this embodiment), of the sheet 2, is referred to as a central region Ra (see FIG. 2). Moreover, a region in the vicinity of the outer peripheral edge 22 of the sheet 2, i.e., a predetermined region in a direction from the outer peripheral edge 22 of the sheet 2 toward the opening 21 (an inner side in the radial direction of the sheet 2, in this embodiment), of the sheet 2, is referred to as a peripheral edge region Rb (see FIG. 2). Furthermore, a region between the central region Ra and the peripheral edge region Rb is referred to as an intermediate region Rc (see FIG. 2). A range of the central region Ra of the sheet 2 is not particularly limited, but can be, for example, a region from the opening edge 21a of the sheet 2 to 30 to 60% of a distance (in the radial direction) between the opening edge 21a and the outer peripheral edge 22 of the sheet 2. Moreover, a range of the peripheral edge region Rb of the sheet 2 is not particularly limited, but can be a region from the outer peripheral edge 22 of the sheet 2 to 20 to 60% of a distance (in the radial direction) between the opening edge 21a and the outer peripheral edge 22 of the sheet 2. Preferably, a range of the central region Ra and the intermediate region Rc can be a region from the opening edge 21a of the sheet 2 to 50% of a distance (in the radial direction) between the opening edge 21a and the outer peripheral edge 22 of the sheet 2.

Moreover, as will be described later, the sheet 2 has (a plurality of) through holes 23 (see FIG. 6) penetrating in the thickness direction of the sheet 2 for inserting the filamentary body 3 therethrough. The through holes 23 are formed at predetermined positions in accordance with a predetermined protruded and recessed structure (by the filamentary body 3) to be formed in the sheet 2, which will be mentioned later, before the filamentary body 3 is sewn. With the through holes 23 provided at predetermined positions in the sheet 2 before the filamentary body 3 is sewn, the through holes 23 serve as markers for positions at which the filamentary body 3 may be sewn, so that a protruded and recessed structure by the filamentary body 3 can be easily formed. In this embodiment, four through holes 23 are provided in a radial direction and aligned in a circumferential direction, but the number and arrangement of the through holes 23 are changed as appropriate depending on a sewing pattern.

A size of the through hole 23 through which the filamentary body 3 is inserted is not particularly limited as long as the filamentary body 3 can be inserted through the through hole 23. In this embodiment, the through hole 23 is formed so that a clearance CL (see FIG. 6) between an inner circumference of a site of the sheet 2 where the through hole 23 is formed and the filamentary body 3 is 1 mm or less, preferably 100 to 300 μm. In this case, a body fluid and a cell become easy to flow toward epidermis not only in a gap between a filamentary body 3, which will be mentioned later, and the surface of the sheet 2, but also in a clearance CL between the filamentary body 3 and the through-holes 23 of the sheet 2, making it easier for the cell of the wall-like tissue T to grow and anchor. The above-described clearance CL can be a maximum gap in an extending direction of the surface of the sheet 2 between the inner circumference (inner surface) of the site of the sheet 2 where the through hole 23 is formed and an outer circumference of the filamentary body 3. It should be noted that a filamentary body 3 may be inserted multiple times or a plurality of filamentary bodies 3 may be inserted through one through hole 23 from one surface 2a (see FIG. 4) to the other surface 2b (see FIG. 4) of the sheet 2, or from the other surface 2b to one surface 2a. In this case, the clearance CL is a maximum gap between the inner circumference (opening edge) of the site of the sheet 2 where the through hole 23 is formed and the outer circumference of the filamentary body 3 in a state in which a filamentary body 3 is inserted therethrough multiple times or a plurality of filamentary bodies 3 is inserted therethrough.

As shown in FIGS. 1 to 6, the fixing structure 1 comprises filamentary body 3 sewn onto the surface of the sheet 2. The filamentary body 3 forms a protruded and recessed structure that provides resistance to a relative movement of the sheet 2 in the extending direction of the sheet 2 with respect to a target site of a living body into which the insertion member IS is inserted. Here, the target site of the living body is a peripheral portion of a site into which the insertion member IS is inserted, in the wall-like tissue T. More specifically, the target site of the living body is a site incised to install the fixing structure 1 into which the insertion member IS is inserted, of the wall-like tissue T. In the present specification, the term “filamentary body” is not limited to a filamentary body sewn only to the sheet 2 (such as a radial sewing part 31, a circumferential sewing part 32, and a sheet-side filamentary body 34, which will be mentioned later), as long as it can form a protruded and recessed structure that provides resistance to a relative movement in the extension direction of the sheet 2 with respect to a target site. For example, the “filamentary body” also includes a fixing filamentary body 33 that fixes the sheet 2 to another member (e.g., a sandwiching part 5 that will be mentioned later), as will be mentioned later.

The “protruded and recessed structure that provides resistance to a relative movement in the extending direction of the sheet 2 with respect to a target site” configured by the filamentary body 3 suppresses a relative movement in the extending direction of the sheet 2, i.e., in any direction perpendicular to the thickness direction of the sheet 2 between the sheet 2 and the target site (wall-like tissue T, where the target site is also referred to as a wall-like tissue T, hereinafter). The protruded and recessed structure is provided by the filamentary body 3 protruding in the thickness direction of the sheet 2 with respect to the surface of the sheet 2 (see FIGS. 1 and 4), with the filamentary body 3 sewn in a predetermined pattern onto the surface of the sheet 2 (one surface 2a and/or the other surface 2b in the thickness direction). More specifically, a part where the filamentary body 3 is sewn to the sheet 2 constitutes a protruded part of the protruded and recessed structure, and a part where the filamentary body 3 is not provided, i.e., the surface of the sheet 2 constitutes a recessed part of the protruded and recessed structure.

As mentioned above, in this embodiment, the fixing structure 1 has the filamentary body 3 sewn onto the surface of the sheet 2 so as to form a protruded and recessed structure that provides resistance to a relative movement in the extension direction of the sheet 2 with respect to a target site. A part of the wall-like tissue T thereby enters into the recessed part of the protruded and recessed structure when the fixing structure 1 is provided on the target site (wall-like tissue T). Moreover, in the fixing structure 1 of this embodiment, the contact area with the wall-like tissue T is larger than that in a fixing structure consisting only of a sheet without a filamentary body, so that the protruded and recessed structure due to the fixing structure 1 also shows a greater resistance when the fixing structure 1 moves relative to the wall-like tissue T. Thus, it is suppressed that the sheet 2 and the wall-like tissue T moves relative to each other (the sheet 2 moves relative to the wall-like tissue T or the wall-like tissue T moves relative to the sheet 2) in the extending direction of the sheet 2 parallel to the surface of the sheet 2 (the radial direction and the circumferential direction of the sheet 2, in this embodiment). Thus, misalignment from the target site can be suppressed without sewing the sheet 2 to the target site such as the skin. Therefore, the fixing structure 1 can be fixed early to the target site by a simple procedure. More specifically, the protruded and recessed structure formed by the filamentary body 3 suppresses the fixing structure 1 from being misaligned relative to the wall-like tissue T of the living body. Accordingly, the cell of the wall-like tissue T proliferates stably and enters into the sheet 2, promoting healing of an incision site (target site) into which the insertion member IS is inserted.

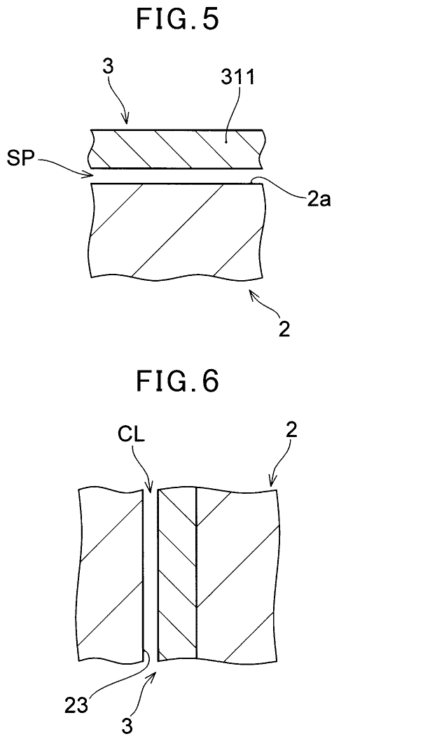

Moreover, by sewing the filamentary body 3 to the sheet 2, as shown in FIG. 5, a minute space SP (in FIG. 5, a size of the space SP is shown to be exaggerated for ease of understanding) is formed between the filamentary body 3 and the sheet 2 in the thickness direction of the sheet 2. When the fixing structure 1 is embedded into the wall-like tissue T and a predetermined period of time has passed, the cell of the wall-like tissue T enters into this minute space SP. The healing of the incision site of the wall-like tissue T is thereby promoted, allowing the fixation structure 1 to be fixed to the wall-like tissue T earlier. Moreover, with the cell of the wall-like tissue T entering into this minute space SP, the filamentary body 3 becomes a constrained state by the entering cell. Thus, even if the entire fixation structure 1 is likely to move relative to the wall-like tissue T, the cell that has entered into the minute space SP becomes entangled with the filamentary body 3, suppressing the relative movement of the fixation structure 1 with respect to the wall-like tissue T.

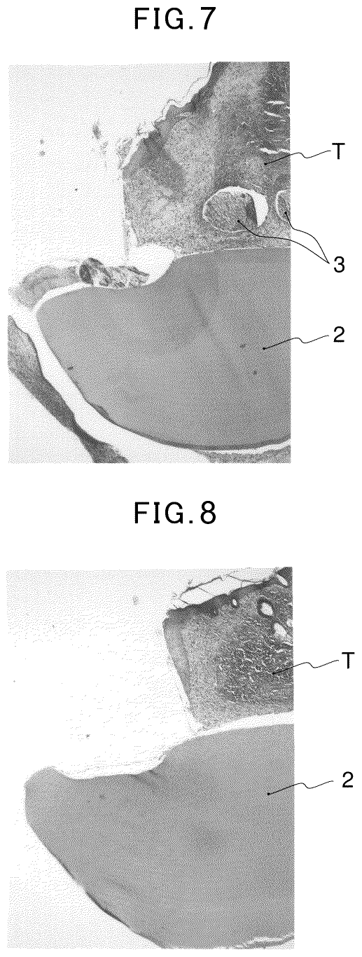

FIG. 7 shows a hematoxylin-eosin staining (HE staining) image obtained by incising the back of a rat and embedding a fixation structure 1 in which filamentary body 3 is sewn to an ePTFE sheet 2 under the skin for 30 days, followed by removing the fixation structure 1 including the skin from the back and magnifying the cross section of the removed part up to 40 times. As shown in FIG. 7, it was observed that the cell of the wall-like tissue T entered between the filamentary body 3 and the sheet 2 and between the filamentary body 3 and the filamentary body 3, making the skin less likely to peel off. Moreover, it was confirmed that the cell that had entered the gap between the filamentary body 3 and the surface of the sheet 2 adhered to the surface of the sheet 2, and the cell entered into the surface of the porous sheet 2 (the surface of the sheet 2 is slightly discolored in the image of FIG. 7). FIG. 8 shows a hematoxylin-eosin staining (HE staining) image obtained by embedding a fixation structure in the same condition as in FIG. 7 except that the fixing structure of Comparative example in which the filamentary body 3 was not sewn was used, followed by removing the fixation structure of Comparative example including the skin from the back and magnifying the cross section of the removed part up to 40 times. In the Comparative example of FIG. 8, a porous ePTFE sheet similar to that in the image of FIG. 7 was used, but the wall-like tissue T peeled off from the surface of the sheet 2, opening the gap, and no intrusion of the cell into the surface of the sheet 2 was observed. As such, it was found that, with the filamentary body 3 sewn to the sheet 2, the cell of the wall-like tissue T enters into the minute space SP between the filamentary body 3 and the sheet 2, promoting the healing of the incision site, and early fixation between the fixing structure 1 and the wall-like tissue T is possible.

The protruded and recessed structure may be provided on at least one surface of the sheet 2, but in this embodiment, the protruded and recessed structure is provided on both surfaces of the sheet 2. When the protruded and recessed structure is provided on both surfaces of the sheet 2, the relative movement between the fixing structure 1 and the wall-like tissue T can be further suppressed, making it easier for the cell of the wall-like tissue T to intrude both the one surface 2a and the other surface 2b of the sheet 2, further promoting the healing of the incision site.

Like the sheet 2, the filamentary body 3 is composed of a non-resorbable material having biocompatibility. The non-resorbable material having biocompatibility that constitutes the filamentary body 3 can be a similar material as that used as a medical suture. The material constituting the filamentary body 3 can be, for example, at least one selected from the group consisting of a polyurethane resin, a polyamide resin, a polylactic acid resin, a polyolefin resin, a polyester resin, a fluororesin, a urea resin, a phenolic resin, an epoxy resin, a polyimide resin, an acrylic resin, a methacrylic resin, and derivatives thereof, and it can be preferably an expanded polytetrafluoroethylene (ePTFE). Moreover, the filamentary body 3 is preferably porous in order to further promote the growth and anchoring of the cell of the wall-like tissue T.

A thickness of the filamentary body 3 is not particularly limited as long as it can form a protruded and recessed structure that provides resistance to the relative movement in the extending direction of the sheet 2 with respect to the target site, and, for example, a thread having a diameter of 0.1 to 0.5 mm, preferably 0.1 to 0.3 mm, and more preferably 0.2 mm can be used.

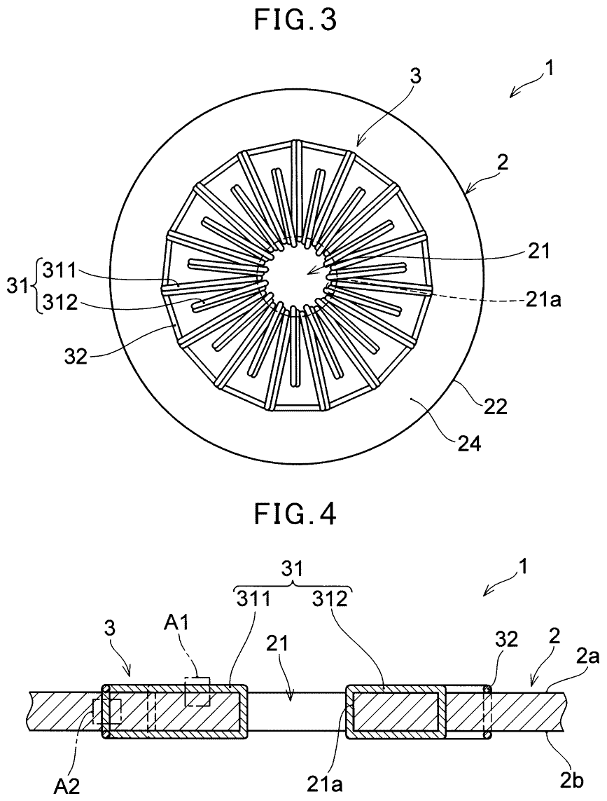

The filamentary body 3 is sewn to the sheet 2 in a predetermined sewing pattern that will be mentioned later. A sewing pattern of the filamentary body 3 is not particularly limited as long as it can form a protruded and recessed structure that provides resistance to the relative movement in the extending direction of the sheet 2 with respect to the target site. The filamentary body 3 may be sewn continuously in the entire sewing pattern with a single filamentary body 3 or may be sewn separately with a plurality of filamentary bodies 3. Moreover, as shown in FIGS. 2 and 3, the filamentary body 3 may be sewn in the same sewing pattern on one surface 2a and the other surface 2b or may be sewn in a different sewing pattern on one surface 2a and the other surface 2b.

Next, the sewing pattern of the filamentary body 3 in this embodiment will be described.

In this embodiment, as shown in FIGS. 2 and 3, the filamentary body 3 has a plurality of radial sewing parts 31 extending in a radiating direction connecting the opening 21 and the outer peripheral edge 22 of the sheet 2. Moreover, the filamentary body 3 also has a circumferential sewing part 32 extending in a circumferential direction of the sheet 2.

The radial sewing part 31 is a part of the sewing pattern of the filamentary body 3 extending radially. Here, “extending radially” means that the filamentary body 3 extends in a direction connecting the opening 21 and the outer peripheral edge 22 (hereinafter referred to as a radial direction). In this embodiment, the radial sewing part 31 extends in a radius direction of a circular sheet 2. It should be noted that the radial sewing part 31 may be inclined with respect to the radius direction of the sheet 2 as long as it extends in a direction connecting the opening 21 and the outer peripheral edge 22 so as to radiate from the opening 21. Moreover, in this embodiment, the radial sewing part 31 extends radially from the opening edge 21a of the sheet 2, but it does not necessarily have to extend from the opening edge 21a, and may extend from a position closer to the outer peripheral edge 22 with respect to the opening edge 21a toward the outer peripheral edge 22 (radially outwardly). In this embodiment, as shown in FIGS. 2 and 3, the radial sewing part 31 extends in a radial direction from the opening 21 with a length that does not reach the outer peripheral edge 22, and as will be mentioned later, the sheet 2 has a non-sewing part 24 to which no filamentary body 3 is sewn.

The radial sewing part 31 constitutes a part of the protruded and recessed structure, and suppresses a relative movement in a direction intersecting in a radial direction between the fixing structure 1 and the wall-like tissue T by extending radially. In this embodiment, the radial sewing part 31 is composed of a plurality of radial sewing parts 311, 312 extending in a plurality of radial directions at predetermined angular intervals from the opening edge 21a. The plurality of radial sewing parts 311, 312 are provided over the entire circumferential direction of the opening edge 21a. When the plurality of radial sewing parts 311, 312 extending in the plurality of radial directions are provided, the relative movement of the fixing structure 1 with respect to the wall-like tissue T can be suppressed even if forces are applied to the fixing structure 1 in various directions. Moreover, even if a force is applied to the fixing structure 1 in a direction rotating the fixing structure 1 in the circumferential direction of the sheet 2 (a direction around an axis of the insertion member IS) with respect to the wall-like tissue T, the respective radial sewing parts 311, 312 provide resistance, so that the rotation of the fixing structure 1 can be suppressed. In this embodiment, the plurality of radial sewing parts 311, 312 extending in the plurality of radial directions are provided with different lengths from each other, but the plurality of radial sewing parts extending in the plurality of radial directions may have the same length.

As mentioned above, the radial sewing part 31 may have a plurality of radial sewing parts 311, 312 having different lengths from each other. In this embodiment, as shown in FIGS. 2 and 3, the radial sewing part 31 has a plurality of first radial sewing parts 311 each extending in a radial direction from the opening 21 with a predetermined length and a plurality of second radial sewing parts 312 each extending in a radial direction from the opening 21 with a length shorter than that of the first radial sewing part 311, though the details will be mentioned later. It should be noted that the radial sewing part 31 may further have other radial sewing parts (e.g., a third radial sewing part, a fourth radial sewing part, etc.) that are different in length from the first radial sewing part 311 and the second radial sewing part 312.

Moreover, as shown in FIGS. 2 and 3, a part of the plurality of radial sewing parts 31 may extend parallel to each other in one radial direction with being adjacent to each other in a circumferential direction. In this case, a space is formed between the adjacent radial sewing parts 31 to each other. When the cell of the wall-like tissue T enters into this space, the cell that has entered into the space becomes entangled with the filamentary body 3, so that the relative movement of the fixing structure 1 with respect to the wall-like tissue T is further suppressed.

The circumferential sewing part 32 is a part of the sewing pattern of the filamentary body 3 extending in a circumferential direction. Here, “extending in a circumferential direction” means extending in substantially the same direction as a direction along the outer peripheral edge 22 of the sheet 2 or the opening edge 21a of the sheet 2. In this embodiment, the circumferential sewing part 32 extends in a direction intersecting in a radial direction (a radius direction of the sheet 2). More specifically, the circumferential sewing part 32 extends so as to connect one radial sewing part 31 (a first radial sewing part 311) to another radial sewing part 31 (a first radial sewing part 311) at the same radial position.

In this embodiment, the circumferential sewing part 32 extends so as to intersect with the radial sewing part 31 (extends so as to connect the ends of the first radial sewing part 311 on the outer peripheral edge 22 side to each other). However, the circumferential sewing part 32 does not necessarily have to intersect with the radial sewing part 31. Moreover, in this embodiment, the circumferential sewing part 32 is provided continuously in a circumferential direction in a circular pattern around the axis of the insertion member IS inserted into the opening 21. However, the circumferential sewing part 32 does not need to be provided continuously in the circumferential direction, but may be interrupted midway and provided intermittently in the circumferential direction.

The circumferential sewing part 32 constitutes a part of the protruded and recessed structure, and suppresses a relative movement in a direction intersecting in a circumferential direction between the fixing structure 1 and the wall-like tissue T by extending in a circumferential direction. With the circumferential sewing part 32 suppressing the relative movement in the direction intersecting in the circumferential direction, the fixing structure 1 embedded into the wall-like tissue T is suppressed from being detached from the wall-like tissue T. For example, when the insertion member IS is pulled upward in FIG. 1, the fixing structure 1 receives a force such that the central portion in the vicinity of the opening 21 is pulled upward. In this case, a part of the fixing structure 1 at right-side with respect to the insertion member IS receives forces to move to the left and a part of the fixing structure 1 at left-side with respect to the insertion member IS receives forces to move to the right side in FIG. 1. The circumferential sewing part 32 catches on the wall-like tissue T to be resistant to this force, thereby suppressing the fixation structure 1 from being misaligned and detaching from the wall-like tissue T. Moreover, as the circumferential sewing part 32 extends continuously in the circumferential direction, bending of the sheet 2 along a bending line in a direction intersecting in the circumferential direction can be suppressed.

Moreover, in this embodiment, the sheet 2 has a non-sewing part 24 to which no filamentary body 3 is sewn in a peripheral edge region Rb having a predetermined width from the outer peripheral edge 22 of the sheet 2, as shown in FIGS. 2 and 3. The non-sewing part 24 is a part where neither a radial sewing part 31 nor a circumferential sewing part 32 is provided, composed only by the sheet 2. With the sheet 2 having the non-sewing part 24, the peripheral edge region Rb of the fixing structure 1 is in a flexible state with respect to the central region Ra. Thus, with the fixing structure 1 embedded into the wall-like tissue T, it can follow movement of the wall-like tissue T such as the skin.

Moreover, in this embodiment, the filamentary body 3 is sewn to the sheet 2 so that the filamentary body 3 changes from a denser state to a sparser state toward the outer peripheral edge 22 side (peripheral edge region Rb side) of the sheet 2 from the central region Ra side around the opening 21 of the sheet 2. In this case, the filamentary body 3 (particularly the radial sewing part 31 in this embodiment) becomes denser in the central region Ra, thereby increasing the rigidity of the central region Ra of the fixing structure 1, which makes the fixing structure 1 less likely to deform in the central region Ra. The deformation of the fixing structure 1 is thereby suppressed, even if a force that deforms the peripheral portion around the opening 21 of the fixing structure 1 is applied from the insertion member IS inserted into the fixing structure 1 due to the weight of the insertion member IS or the like. Thus, the insertion member IS inserted into the fixing structure 1 can be maintained at a predetermined angle with respect to the target site (wall-like tissue T). Thus, the grown cell is suppressed from being peeled off between the fixing structure 1 and the wall-like tissue T due to the fixing structure 1 being deformed or tilted with respect to the wall-like tissue T. Therefore, the cell growth is not inhibited, and the cell proliferation in the central region Ra becomes active, so that the healing of the incision site is promoted, and downgrowth can be suppressed.

Moreover, the filamentary body 3 becomes sparser on the peripheral edge region Rb side of the sheet 2, so that the fixing structure 1 (sheet 2) follows the movement of the target site such as the skin and conforms to a shape of the target site such as the skin. Thus, a patient having the fixation structure 1 implanted therein can feel less discomfort when the fixation structure 1 is implanted. It should be noted that “changing from a denser state to a sparser state toward the outer peripheral edge 22 side from the central region Ra side” means that an amount of the filamentary body 3 per unit area in a predetermined entire region on the central region Ra side (the entire region within the central region Ra in FIG. 2) is greater than an amount of the filamentary body 3 per unit area in a predetermined entire region on the outer peripheral edge 22 side (the entire intermediate region Rc or the entire peripheral edge region Rb in FIG. 2). Thus, “changing from a denser state to a sparser state toward the outer peripheral edge 22 side from the central region Ra side” refers to a comparison between predetermined regions having predetermined widths in the radial direction, and even if the filamentary body 3 has a part where a density of the filamentary body 3 (a proportion occupied by the filamentary body 3 per unit area of the fixing structure 1) increases locally as it moves to the outer side in the radial direction, the density only needs to be lower when compared for each region.

A sewing pattern of the filamentary body 3 is not particularly limited as long as the filamentary body 3 is sewn to the sheet 2 so that it changes from a denser state to a sparser state toward the outer peripheral edge 22 side from the central region Ra side. In this embodiment, the radial sewing part 31 has a first radial sewing part 311 and a second radial sewing part 312, and an end on the outer peripheral edge side of the first radial sewing part 311 (hereinafter referred to as an outer end) is located on an outer side in the radial direction with respect to an outer end of the second radial sewing part 312. The first radial sewing part 311 thereby protrudes in the radial direction to the second radial sewing part 312. Thus, the radial sewing part 31 has a higher density in the central region Ra provided with both the first radial sewing part 311 and the second radial sewing part 312 than in the intermediate region Rc not provided with the second radial sewing part 312 (note that, in this embodiment, the circumferential sewing part 32 is provided in the intermediate region Rc, but the central region Ra is denser than the intermediate region Rc, even including the circumferential sewing part 32).

Moreover, as shown in FIG. 1, a predetermined region of the central region Ra from the opening edge 21a of the opening 21 may be sandwiched by connecting members C, etc., and in such a case, a part where the filamentary body 3 changes from a denser state to a sparser state only needs to change from a denser state to a sparser state in a part not sandwiched by the connecting members C, etc.

Moreover, it is preferable that the filamentary body 3 (particularly the radial sewing part 31) stepwisely changes from a denser state to a sparser state toward the outer peripheral edge 22 side from the central region Ra side. “Stepwisely changing from a denser state to a sparser state” means that the filamentary body 3 has a region with two or more stages of different density, but it is preferable that the filamentary body 3 is stepwisely provided with a region with three or more stages of different density.

When the filamentary body 3 is sewn so as to stepwisely change from a denser state to a sparser state, the fixing structure 1 can easily follow the movement of the target site such as the skin from the central region Ra to the peripheral edge region Rb of the sheet 2. Therefore, the sheet 2 is suppressed from suddenly changing in rigidity and being locally applied with stress, and the sheet 2 is suppressed from bending to cause the part to which the surface of the sheet 2 and the cell of the wall-like tissue T are fixed to peel off, suppressing delayed healing of the incision site.

Next, fixing structures of other embodiments will be described. It should be noted that in the following descriptions, descriptions of matters common to the first embodiment mentioned above will be omitted, focusing on the differences. It should be noted that all of the matters described in the first embodiment can be applied to fixing structures of other embodiments as long as the purpose of the invention can be achieved, and the following configurations of other embodiments below and the contents described in the first embodiment can be used in combination. Moreover, the effects obtained by the configuration described in the first embodiment can also be obtained in other embodiments below as long as the configuration is included. In addition, the configurations of other embodiments below can be combined with each other.

### Second Embodiment

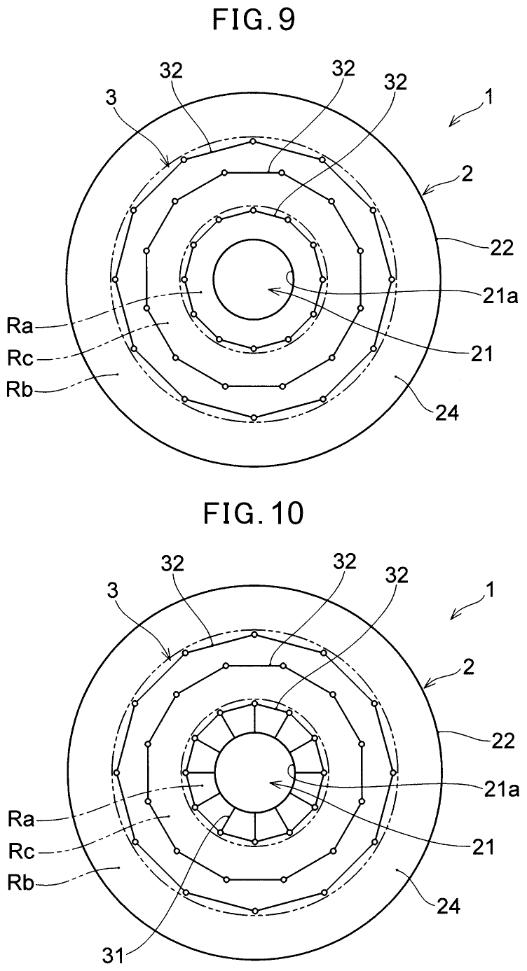

As shown in FIG. 9, the fixing structure 1 of the second embodiment has a plurality of circumferential sewing parts 32. Specifically, the fixing structure 1 of the second embodiment has a plurality of (three in FIG. 9) circumferential sewing parts 32 with different distances from the opening edge 21a in a radial direction. The fixing structure 1 of this embodiment does not have any radial sewing part, and has a non-sewing part 24 that does not have any filamentary body 3 in the peripheral edge region Rb. In this embodiment, with the plurality of circumferential sewing parts 32 provided, the relative movement in the radial direction between the fixing structure 1 and the wall-like tissue T is further suppressed.

### Third Embodiment

As shown in FIG. 10, the fixing structure 1 of the third embodiment has a plurality of (three in FIG. 10) circumferential sewing parts 32 with different distances from the opening edge 21a in the radial direction, similarly to the second embodiment. The fixing structure 1 of this embodiment has a non-sewing part 24 that does not have any filamentary body 3 in the peripheral edge region Rb. Moreover, in this embodiment, the fixing structure 1 has a plurality of radial sewing parts 31 that are provided in the central region Ra and extend in a radial direction from a circumferential sewing part 32 closest to the opening 21 toward the opening edge 21a. With the plurality of circumferential sewing parts 32 provided in the fixing structure 1 of this embodiment, the relative movement in the radial direction between the fixing structure 1 and the wall-like tissue T is further suppressed, similarly to the second embodiment. Moreover, the fixing structure 1 has a plurality of radial sewing parts, so that the relative movement in the direction intersecting in the radial direction between the fixing structure 1 and the wall-like tissue T can be suppressed. Furthermore, since the plurality of radial sewing parts 31 are provided over the entire circumferential direction of the opening edge 21a, rotation of the fixing structure 1 relative to the wall-like tissue T can be suppressed. In addition, since the central region Ra side is denser and the outer peripheral edge 22 side of the sheet 2 is sparser, the rigidity of the central region Ra of the fixing structure 1 is increased, and the fixing structure 1 becomes less likely to deform in the central region Ra. Thus, as mentioned above, the insertion member IS inserted into the fixing structure 1 can be maintained at a predetermined angle with respect to the target site (wall-like tissue T). Thus, the grown cell is suppressed from being peeled off between the fixing structure 1 and the wall-like tissue T due to the fixing structure 1 being deformed or tilted with respect to the wall-like tissue T. Therefore, the cell growth is not inhibited, and the cell proliferation in the central region Ra becomes active, so that the healing of the incision site is promoted, and downgrowth can be suppressed.

It should be noted that when the part of the central region Ra around the opening 21 is sandwiched by connecting members C, etc., as mentioned above, the circumferential sewing part 32 and the radial sewing parts 31 extending on an inner side in the radial direction from the circumferential sewing parts 32 can be provided to be shifted to the outer peripheral edge 22 side in the radial direction from the part sandwiched by the connecting members C, etc.

### Fourth Embodiment

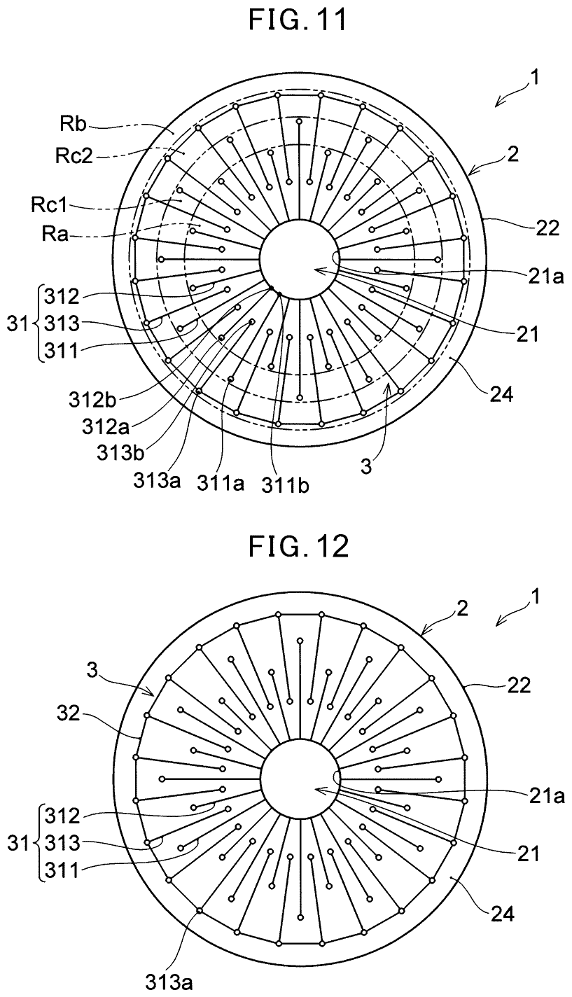

As shown in FIG. 11, the fixing structure 1 of the fourth embodiment has a plurality of different radial sewing parts 31 extending from the central region Ra toward the outer peripheral edge 22 side. The fixing structure 1 has a plurality of radial sewing parts 31, so that the relative movement in the direction intersecting in the radial direction between the fixing structure 1 and the wall-like tissue T can be suppressed. Furthermore, since the plurality of radial sewing parts 31 are provided over the entire circumferential direction of the opening edge 21a, rotation of the fixing structure 1 relative to the wall-like tissue T can be suppressed. In this embodiment, no circumferential sewing part is provided. It should be noted that the two-dot chain lines in FIG. 11 indicate boundaries of the region of the fixing structure 1.

Moreover, in this embodiment, the plurality of different radial sewing parts 31 are sewn such that positions of outer ends 311a, 312a, 313a, which are ends on the outer peripheral edge 22 side, are different from one another. In this embodiment, with the positions of the outer ends 311a, 312a, 313a of the radial sewing parts 31 extending from the central region Ra toward the outer peripheral edge 22 side being different from one another, the fixing structure 1 is configured to stepwisely change from a denser state to a sparser state from the central region Ra side toward the outer peripheral edge 22 side.

Specifically, the fixing structure 1 of this embodiment comprises a plurality of first radial sewing parts 311, a plurality of second radial sewing parts 312, and a third radial sewing part 313 extending from the first radial sewing parts 311 toward the outer peripheral edge 22 side (the outer side in the radius direction) of the sheet 2. More specifically, the first to third radial sewing parts 311, 312, 313 are configured such that the outer end 312a of the second radial sewing part 312 is positioned on the innermost in the radial direction (the opening 21 side), the outer end 313a of the third radial sewing part 313 is positioned on the outermost in the radial direction (the outer peripheral edge 22 side), and the outer end 311a of the first radial sewing part 311 is positioned between the outer end 312a of the second radial sewing part 312 and the outer end 312a of the third radial sewing part 313 in the radial direction. The fixing structure 1 of this embodiment thereby becomes a denser state where the density of the radial sewing part 31 is highest in the central region Ra, and the density of the radial sewing part 31 gradually approaches to a sparser state toward the outer peripheral edge 22 side. More specifically, in this embodiment, as shown in FIG. 11, the fixing structure 1 has a first region (central region) Ra in which the first to third radial sewing parts 311, 312, 313 are sewn and which has the highest density of the radial sewing part 31, a second region (first intermediate region) Rc1 which is adjacent to the outer peripheral edge 22 side of the first region, in which the first and third radial sewing parts 311, 313 are sewn, and which has the second highest density after the first region Ra, a third region (second intermediate region) Rc2 which is adjacent to the outer peripheral edge 22 side of the second region Rc1, in which the third radial sewing part 313 is sewn, and which has the third highest density after the second region Rc1, and a fourth region (peripheral edge region) Rb which is the sparsest and is a non-sewing part 24 to which no radial sewing part 31 is sewn. It should be noted that the number of different radial sewing parts 31 can be changed as appropriate, for example, the fixing structure 1 may have a fourth radial sewing part, a fifth radial sewing part, and the like.

As mentioned above, the fixing structure 1 is configured to stepwisely change from a denser state to a sparser state from the central region Ra side toward the outer peripheral edge 22 side. The rigidity of the central region Ra of the fixing structure 1 is thereby increased, and the fixing structure 1 becomes less likely to deform in the central region Ra. Thus, as mentioned above, the insertion member IS inserted into the fixing structure 1 can be maintained at a predetermined angle with respect to the target site (wall-like tissue T). Thus, the grown cell is suppressed from being peeled off between the fixing structure 1 and the wall-like tissue T due to the fixing structure 1 being deformed or tilted with respect to the wall-like tissue T. Therefore, the cell growth is not inhibited, and the cell proliferation in the central region Ra becomes active, so that the healing of the incision site is promoted, and downgrowth can be suppressed. Moreover, in this embodiment, as mentioned above, a filamentary body 3 is sewn by a plurality of different radial sewing parts 31 so that it stepwisely changes from a denser state to a sparser state. In this case, the fixing structure 1 can easily follow the movement of the target site such as the skin from the central region Ra to the peripheral edge region Rb of the sheet 2. Therefore, the sheet 2 is suppressed from suddenly changing in rigidity and being locally applied with stress, and the sheet 2 is suppressed from bending to cause the part to which the surface of the sheet 2 and the cell of the wall-like tissue T are fixed to peel off, suppressing delayed healing of the incision site.

In this embodiment, the third radial sewing part 313 may extend to the opening edge 21a. Moreover, the periphery of the opening 21 in the central region Ra may be sandwiched by connecting members C, etc. (see FIG. 1), in which case the inner ends 311b, 312b of the first and second radial sewing parts 311, 312 may be located on the outer peripheral edge 22 side relative to the opening 21 (for example, at the same position in the radius direction as the inner end 313b of the third radial sewing part 313).

### Fifth Embodiment

The fixing structure 1 of the fifth embodiment further has a circumferential sewing part 32 compared to the fixing structure 1 of the fourth embodiment. In the fifth embodiment, in addition to the effects described in the fourth embodiment, the circumferential sewing part 32 can suppress the relative movement in the radial direction between the fixing structure 1 and the wall-like tissue T.

In this embodiment, the circumferential sewing part 32 extends in a circumferential direction so as to connect the outer ends 313a of the plurality of third radial sewing parts 313, but it should be noted that the position of the circumferential sewing part 32 in the radius direction is not limited to the position shown in the figure. Moreover, as shown in FIG. 9, a plurality of circumferential sewing parts 32 may be provided at a plurality of different radial positions.

Moreover, as a further variation, the sheet 2 used in any of the above-mentioned embodiments may be configured so that the flexibility of the sheet 2 gradually increases from the central region Ra around the opening 21 of the sheet 2 toward the outer peripheral edge 22 of the sheet 2. In this case, the sheet 2 can easily follow the movement of the target site such as the skin from the central region Ra to the peripheral edge region Rb, suppressing the rigidity of the sheet 2 from suddenly changing to cause a stress to be locally applied. Thus, a problem becomes less likely to occur, in which the sheet 2 bends due to the local stress to cause the part to which the surface of the sheet 2 and the cell of the wall-like tissue T are fixed to peel off, etc., leading to delayed healing. It should be noted that a configuration for gradually increasing the flexibility of the sheet 2 from the central region Ra toward the outer peripheral edge 22 is not particularly limited, but for example, the flexibility of the sheet 2 can be gradually increased from the central region Ra toward the outer peripheral edge 22 by changing the thickness of the sheet 2 toward the outer peripheral edge 22 of the sheet 2 or by applying a coating to the sheet 2.

### Sixth Embodiment

The fixing structure 1 of the sixth embodiment differs from the fixing structures of the first to fifth embodiments in that it comprises a cylindrical insertion part 4 that guides the insertion member IS and a sandwiching part 5 for sandwiching the sheet 2 and being fixed to the insertion part 4, as will be mentioned later. It should be noted that the points described below can also be applied to the fixing structures of the first to fifth embodiments and the seventh to ninth embodiments.

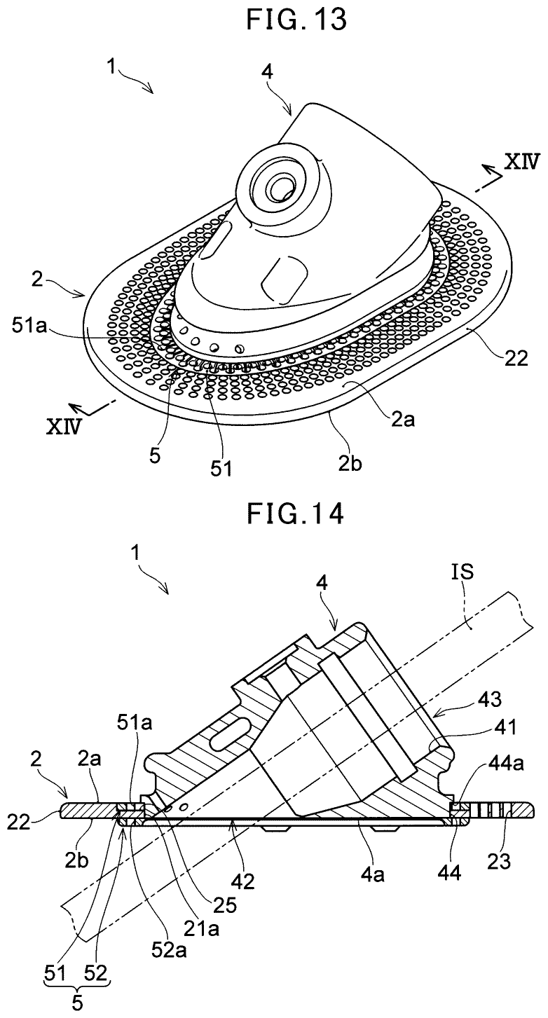

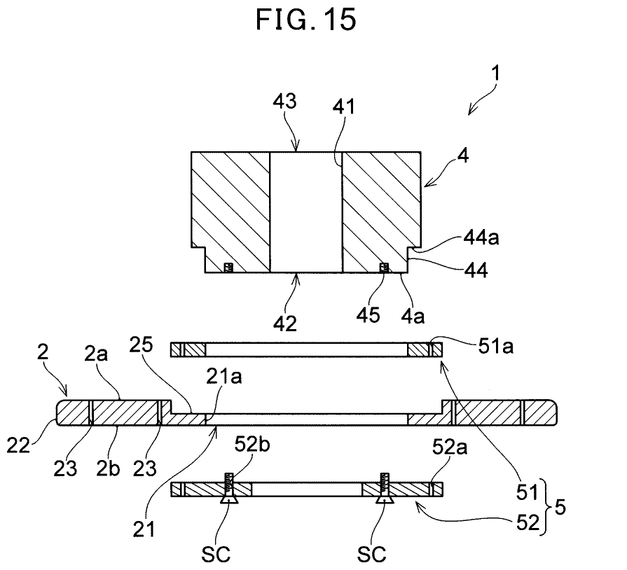

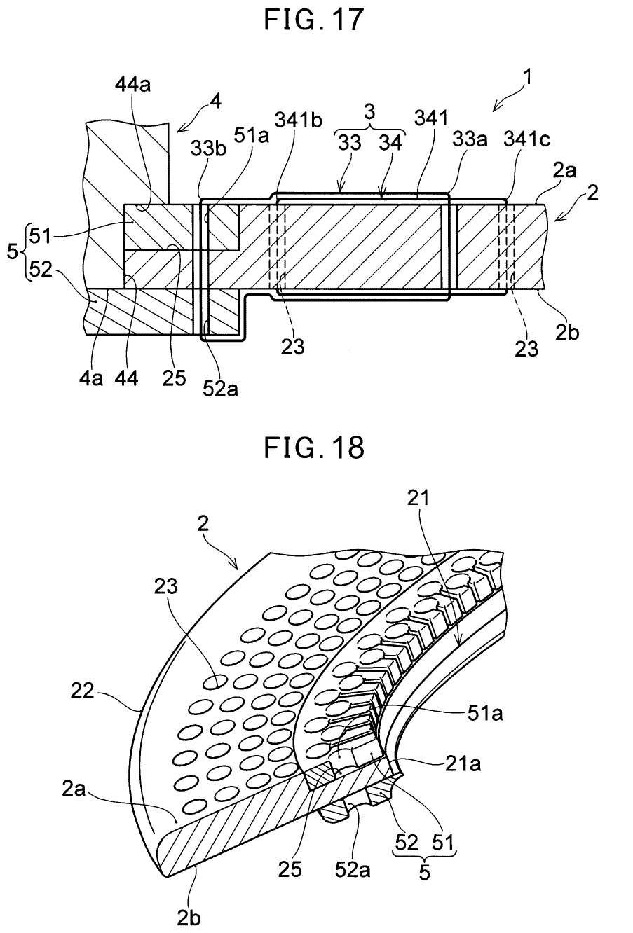

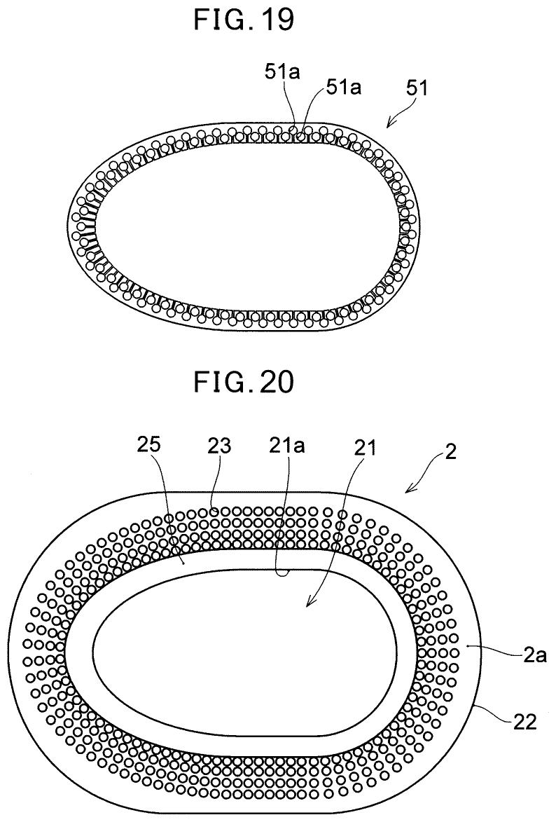

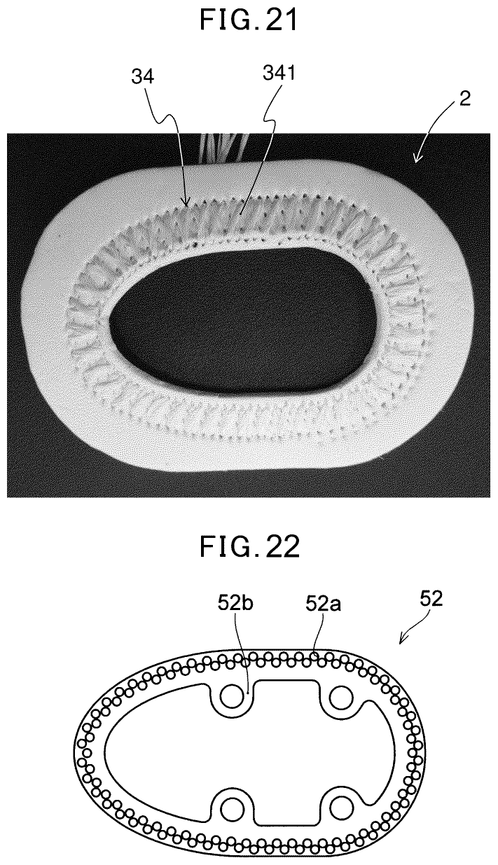

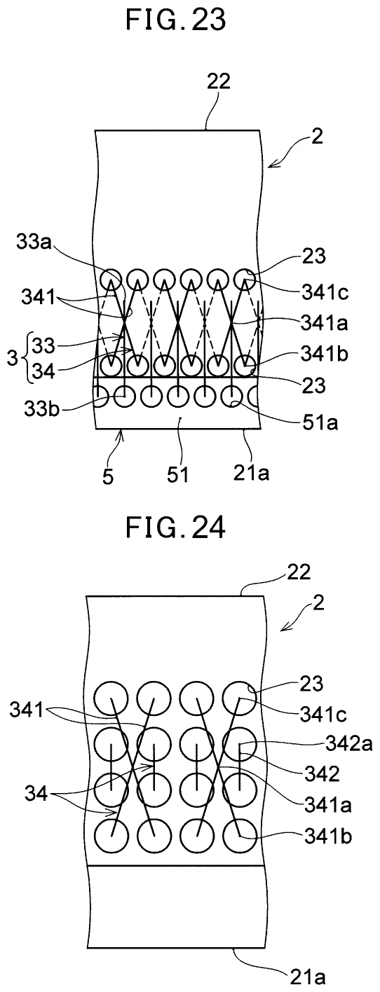

FIG. 13 is a perspective view of the fixing structure of the sixth embodiment before a filamentary body is sewn thereon. FIG. 14 is a cross-sectional view taken along a line XIV-XIV in FIG. 13. FIG. 15 is an exploded view showing a simplified fixing structure of the sixth embodiment. FIG. 16 is a schematic view showing a state where respective members of the fixing structure are assembled to each other and the filamentary body is sewn from the state shown in FIG. 15. FIG. 17 is an enlarged view of a part of the fixing structure in FIG. 16 where the filamentary body is sewn. It should be noted that FIGS. 15 to 17 show a conceptually simplified view of the fixing structure, where the number of through holes, shapes of insertion parts, etc. do not match the fixing structure shown in FIG. 14. FIG. 18 is a view showing a state where a sandwiching part is mounted to a sheet in the sixth embodiment. FIG. 19 is a plan view showing a first sandwiching member of the sandwiching part in the sixth embodiment. FIG. 20 is a plan view of a sheet in the sixth embodiment. FIG. 21 is a photograph showing a state where a sheet-side filamentary body is sewn to a sheet in the sixth embodiment. FIG. 22 is a plan view showing a second sandwiching member of the sandwiching part in the sixth embodiment. FIG. 23 is a schematic view showing a simplified one example of a sewing pattern in which a fixing filamentary body and a sheet-side filamentary body are sewn.

As shown in FIGS. 13 to 18, the fixing structure 1 of the present embodiment comprises a cylindrical insertion part 4 having an insertion passage 41 for guiding an insertion member IS (see FIG. 14) from inside of the living body to outside of the living body, and a sandwiching part 5 for sandwiching a peripheral portion around an opening of a sheet 2 in a thickness direction of the sheet 2 and being fixed to the insertion part 4.

The insertion part 4 supports the insertion member IS with respect to a living body in a state where the insertion member IS is inserted into the insertion part 4. The insertion part 4 is connected to the sheet 2 as will be mentioned later. In the present embodiment, the insertion part 4 is connected to the sheet 2 via the sandwiching part 5.

The insertion part 4 communicates between inside of the living body and outside of the living body. In the present embodiment, the insertion part 4 is a part where an insertion member IS such as a drive line is inserted through, and is configured in a substantially cylindrical shape. As shown in FIGS. 14 to 16, the insertion part 4 has a first opening 42 on the inside of the living body side and a second opening 43 on the outside of the living body side. The insertion passage 41 is formed between the first opening 42 and the second opening 43.

A shape and a structure of the insertion part 4 are not particularly limited as long as the insertion part 4 can support the insertion member IS with respect to a living body in a state where the insertion member IS is inserted into the insertion part 4. In the present embodiment, the insertion part 4 is configured to protrude to the outside of the living body side. Specifically, as shown in FIG. 14, the insertion part 4 is configured such that an axis of the insertion passage 41 is inclined with respect to the surface of the sheet 2, but an inclination angle of the axis of the insertion passage 41 is not particularly limited. Moreover, the insertion passage 41 may be provided so as to be orthogonal to the sheet 2 (see the schematic view of FIG. 16, etc.).

Although not shown in the figures, the insertion part 4 may have a chuck member that is arranged between the inner wall of the insertion passage 41 and the outer surface of the insertion member IS and tightens and grips the outer peripheral surface of the insertion member IS, a sealing member for holding the insertion member IS liquid tightly within the insertion passage 41 in the insertion part 4, and the like. The chuck member the sealing member, and the like, which are provided between the inner wall of the insertion passage 41 and the outer surface of the insertion member IS, can be of known configurations, and therefore detailed descriptions thereof are omitted. It should be noted that a material constituting the insertion part 4 is composed of a material having biocompatibility and a predetermined rigidity. For example, as the material constituting the insertion part 4, a metal material having biocompatibility and a high corrosion resistance, such as titanium and a titanium alloy.

In the present embodiment, as shown in FIGS. 15 to 17, the insertion part 4 has a mounting part 44 to which the peripheral portion around the opening of the sheet 2 and an inner peripheral portion of a first sandwiching member 51 that will be mentioned later are mounted.

The mounting part 44 has an engagement step 44a configured to engage with a surface of the first sandwiching member 51 opposite to a surface that comes into contact with the sheet 2 in the thickness direction of the sheet 2.

The mounting part 44 is a part where a part of the peripheral portion around the opening of the sheet 2 and the inner peripheral portion of the first sandwiching member 51 are arranged when the sheet 2 and the first sandwiching member 51 are mounted to the insertion part 4. The mounting part 44 is configured so that the sheet 2 and the first sandwiching member 51 can be externally fitted to the end of the insertion part 4 where the first opening 42 is provided. In the present embodiment, as shown in FIGS. 14 to 17, the cylindrical part in which the mounting part 44 is provided is configured to be one size smaller in a radius direction than a cylindrical part (second cylindrical part) adjacent in an axial direction (thickness direction of the sheet 2) to the cylindrical part in which the mounting part 44 is provided (first cylindrical part). Due to the difference in size in a radius direction between the first cylindrical part on which the mounting part 44 is provided and the second cylindrical part adjacent to the first cylindrical part, the mounting part 44 is provided with an engagement step 44a that forms a step.

As shown in FIG. 17, with the sheet 2 and the first sandwiching member 51 mounted to the insertion part 4, the engagement step 44a engages with the surface of the first sandwiching member 51 opposite to the surface that comes into contact with the sheet 2 (the surface on the outside of the living body side) in the thickness direction of the sheet 2. As will be mentioned later, the engagement step 44a engages with the first sandwiching member 51 when a force is applied to the sheet 2 from the second sandwiching member 52 toward the first sandwiching member 51 for sandwiching the sheet 2. The sheet 2 is thereby sandwiched with a pressure applied and stably held against the insertion part 4. A shape and a structure of the engagement step 44a are not particularly limited as long as the first sandwiching member 51 and the engagement step 44a can engage with each other. In the present embodiment, the engagement step 44a is configured by an annular flat part that partially faces the surface of the first sandwiching member 51 on the end side where the first opening 42 is provided.

In the present embodiment, a length from the engagement step 44a to an end face 4a of the insertion part 4 having the first opening 42, that is a length of the mounting part 44 in the thickness direction of the sheet 2, is configured to be smaller than a sum of a thickness of the first sandwiching member 51 and a thickness of the sheet 2 (a thickness of the sheet 2 in an arrangement part 25 that will be mentioned later). In this case, the sheet 2 is compressed by the first sandwiching member 51 and the second sandwiching member 52, thereby suppressing gaps from being created between the sheet 2 and the first sandwiching member 51, and between the sheet 2 and the second sandwiching member 52, so that the sheet 2 can be liquid tightly connected to the insertion part 4.

As shown in FIG. 15, the insertion part 4 has a fixing part 45 to which the sandwiching part 5 is fixed. In the present embodiment, the fixing part 45 is provided on the end where the first opening 42 of the insertion part 4 is provided (the end face 4a in the present embodiment). A fixing means between the insertion part 4 and the sandwiching part 5 is not particularly limited as long as it can fix the sandwiching part 5 to the insertion part 4 with the sheet 2 sandwiched by the sandwiching part 5. In the present embodiment, the fixing means between the insertion part 4 and the sandwiching part 5 is a fastening member SC such as a screw and a bolt, and the fixing part 45 is configured to be able to fasten the fastening member SC. In the present embodiment, the fixing part 45 is a screw hole provided on the end face 4a where the first opening 42 of the insertion part 4 is provided. When the fastening member SC is fastened through the second sandwiching member 52, the second sandwiching member 52 moves in a direction approaching the first sandwiching member 51, and the sandwiching parts 5 are fixed to the insertion part 4 with the sheet 2 compressed and sandwiched. By fixing the sandwiching part 5 to the insertion part 4 with the sheet 2 sandwiched by the sandwiching part 5, it is possible to compress the sheet 2 uniformly, to suppress gaps from being created between the insertion part 4 and the sheet 2, to increase rigidity of a region (arrangement part 25) on the opening edge 21a side of the sheet 2, and to prevent the sheet 2 from slipping out of the insertion part 4.

As shown in FIGS. 14, 16 and 17, the sandwiching part 5 sandwiches the peripheral portion around the opening of the sheet 2 in the thickness direction of the sheet 2. Moreover, as shown in FIG. 16, the sandwiching part 5 is fixed to the insertion part 4 for indirectly fixing the sandwiched sheet 2 to the insertion part 4. In the present embodiment, as shown in FIGS. 14 to 17, the sandwiching part 5 comprises a first sandwiching member 51 that is annular and comes into contact with one surface 2a of the sheet 2 in the peripheral portion around the opening of the sheet 2 and a second sandwiching member 52 that is annular and comes into contact with the other surface 2b of the sheet 2 in the peripheral portion around the opening of the sheet 2.

The first sandwiching member 51, together with the second sandwiching member 52, sandwiches the sheet 2. One surface of the first sandwiching member 51 comes into contact and engages with the engagement step 44a of the insertion part 4, and the other surface of the first sandwiching member 51 comes into contact with one surface 2a of the sheet 2. The first sandwiching member 51 is composed of a hard biocompatible material having a predetermined rigidity higher than that of the sheet 2, and is arranged to surround the periphery of the insertion part 4 (mounting part 44) as shown in FIGS. 14, 16 and 17.

A material constituting the first sandwiching member 51 is not particularly limited as long as it has biocompatibility and a predetermined rigidity higher than that of the sheet 2. For example, as the material constituting the first sandwiching member 51, a metal material having biocompatibility and a high corrosion resistance, such as titanium and a titanium alloy. When titanium or a titanium alloy is used as the material of the first sandwiching member 51, bacteria are less likely to grow than in the sheet 2, so that intrusion of bacteria along the first sandwiching member 51 toward inside of the living body can be suppressed. More specifically, bacteria that have reached the surface of the first sandwiching member 51 from the outer surface of the insertion part 4 are less likely to proliferate along a surface made of titanium or a titanium alloy. Moreover, even if bacteria proliferate along the surface of the first sandwiching member 51 toward the sheet 2 (the right side in FIG. 17), cell growth is promoted on the surface of the sheet 2, so that a further proliferation of the bacteria is suppressed by the cell growth. Furthermore, a path toward the other surface 2b of the sheet 2 (path through a gap between the opposing members) forms a bypass path by providing the mounting part 44 and the arrangement part 25 in a stepped and complexed manner, except for through holes 23, 51a, etc. through which the filamentary body 3 penetrates. Thus, bacteria are less likely to proliferate and reach the other surface 2b side of the sheet 2. Accordingly, healing of the incision site (target site) is promoted.

A shape of the first sandwiching member 51 is not particularly limited as long as it can be mounted to the insertion part 4 and can sandwich the sheet 2 together with the second sandwiching member 52. In this embodiment, as shown in FIGS. 13, 18 and 19, the first sandwiching member 51 is formed in a substantially elliptical (or substantially oval) shape having a space inside. The first sandwiching member 51 is formed in a thin plate shape having one surface and the other surface. As shown in FIGS. 18 and 19, the first sandwiching member 51 has a plurality of through holes 51a formed in advance for sewing the fixing filamentary body 33 along the circumferential direction of the first sandwiching member 51.

As shown in FIGS. 17 and 18, the first sandwiching member 51 is arranged in an arrangement part 25 provided in the peripheral portion around the opening of the sheet 2. The arrangement part 25 of the sheet 2 is provided with a width corresponding to a width of the first sandwiching member 51 in a direction connecting the opening edge 21a of the opening 21 and the outer peripheral edge 22 of the sheet 2. The first sandwiching member 51 and the opening edge 21a of the sheet 2 are aligned and can be thereby mounted to the mounting part 44 of the insertion part 4 without any gaps, as shown in FIGS. 17 and 18. Moreover, a thickness of the sheet 2 in the arrangement part 25 is configured to be thinner than a thickness of the sheet 2 outside the arrangement part 25 (the outer peripheral edge 22 side of the sheet 2). In the present embodiment, a difference between a thickness of the sheet 2 in parts other than the arrangement part 25 of the sheet 2 and the thickness of the sheet 2 in the arrangement part 25 corresponds to a thickness of the first sandwiching member 51.

The second sandwiching member 52, together with the first sandwiching member 51, sandwiches the sheet 2. The second sandwiching member 52 is fixed to the insertion part 4 as shown in FIG. 16. The sheet 2 sandwiched between the first sandwiching member 51 and the second sandwiching member 52 is thereby indirectly fixed to the insertion part 4. In the present embodiment, the second sandwiching member 52 is fastened to the insertion part 4 so as to move in a direction approaching the first sandwiching member 51 in the thickness direction of the sheet 2, so that the sheet 2 and the first sandwiching member 51 are compressed and sandwiched between the engagement step 44a and the second sandwiching member 52. The sheet 2 is thereby stably fixed to the insertion part 4 via the first sandwiching member 51 and the second sandwiching member 52. Thus, when the sheet 2 is fixed to a target site of a living body, the insertion part 4 becomes easily maintained at a predetermined angle with respect to the sheet 2.

A material constituting the second sandwiching member 52 is not particularly limited as long as it has biocompatibility and a predetermined rigidity higher than that of the sheet 2. For example, as the material constituting the second sandwiching member 52, a metal material having biocompatibility and a high corrosion resistance, such as titanium and a titanium alloy.

A shape of the second sandwiching member 52 is not particularly limited as long as it can be fixed to the insertion part 4 and can sandwich the sheet 1 together with the first sandwiching member 51. In the present embodiment, as shown in FIGS. 18 and 22, the second sandwiching member 52 is formed in a substantially elliptical (or substantially oval) shape having a space inside. The second sandwiching member 52 is formed in a thin plate shape having one surface and the other surface. As shown in FIG. 22, the second sandwiching member 52 has a plurality of through holes 52a formed in advance for sewing the fixing filamentary body 33 along the circumferential direction of the second sandwiching member 52. The through holes 52a of the second sandwiching member 52 are provided at positions corresponding to positions of the through holes 51a of the first sandwiching member 51. Moreover, as shown in FIG. 22, the second sandwiching member 52 has an insertion part 52b for inserting a fastening member SC such as a screw and a bolt. In the present embodiment, a plurality of insertion parts 52b protrude inward from an annular part of the second sandwiching member 52. As will be mentioned later, with the sheet 2 and the first sandwiching member 51 mounted to the mounting part 44 of the insertion part 4, the second sandwiching member 52 is fixed to the insertion part 4 by a fastening member SC inserted into the insertion part 52b. A surface of the second sandwiching member 52 facing the other surface 2b of the sheet 2 thereby presses the sheet 2 toward the first sandwiching member 51, so that the sheet 2 is compressed and held in the sandwiching part 5.

As shown in FIGS. 16, 17 and 23, the fixing structure 1 has a fixing filamentary body 33 composed of a filamentary body sewn to the sheet 2 so as to fix the sheet 2 and the sandwiching part 5. The fixing filamentary body 33 is sewn to the surface of the sheet 2 so as to form a protruded and recessed structure providing resistance to a relative movement of the sheet 2 in the extending direction of the sheet 2 with respect to a target site of a living body where the insertion member IS is inserted. As the fixing filamentary body 33 forms a protruded and recessed structure providing resistance to a relative movement with respect to the target site as mentioned above, a part of the wall-like tissue T enters into the recessed part of the protruded and recessed structure when the fixing structure 1 is provided in the target site (wall-like tissue T). Thus, the sheet 2 and the wall-like tissue T are suppressed from moving relative to each other in the extending direction of the sheet 2 parallel to the surface of the sheet 2. Thus, misalignment from the target site can be suppressed without sewing the sheet 2 to the target site such as the skin. Therefore, the fixing structure 1 can be fixed early to the target site by a simple procedure.

In the present embodiment, the fixing structure 1 has a sheet-side filamentary body 34 besides the fixing filamentary body 33, but it should be noted that it may have only the fixing filamentary body 33 without having any sheet-side filamentary body 34. Moreover, the fixing structure 1 may have only the sheet-side filamentary body 34 without having the fixing filamentary body 33.

The filamentary body constituting the fixing filamentary body 33 can be the same as that in the first embodiment. The fixing filamentary body 33 is sewn to the sheet 2 and the sandwiching part 5 in order to fix the sheet 2 to the sandwiching part 5. In the embodiment, as shown in FIG. 17, the outer end 33a of the fixing filamentary body 33 is inserted into the through hole 23 of the sheet 2 located on the outer side with respect to the arrangement part 25 of the sheet 2, and the inner end 33b of the fixing filamentary body 33 is inserted into the through hole 23 provided in the sheet 2 and the through holes 51a, 52a provided in the sandwiching part 5, sewing the fixing filamentary body 33 to the sheet 2. It should be noted that the through hole 23 into which the fixing filamentary body 33 is inserted only needs to be able to insert the fixing filamentary body 33, and can be formed when sewing the fixing filamentary body 33 to the sheet 2 with a sewing needle or can be formed in the sheet 2 in advance. Moreover, the through hole 23 into which the fixing filamentary body 33 is inserted may be a common through hole with the sheet-side filamentary body 34 or may be a separate through hole, depending on a size of the filamentary body and a size of the sheet. Since there is no need to form a through hole in the sheet 2 in advance by forming a through hole 23 when sewing the fixing filamentary body 33 to the sheet 2 with a sewing needle, the through hole 23 can be easily formed.

The fixing filamentary body 33 is radially sewn as shown in FIGS. 17 and 23. A length of the fixing filamentary body 33 in the radiating direction is not particularly limited. The fixing filamentary body 33 is preferably sewn so that, for example, the outer end 33a of the fixing filamentary body 33 is located in a region from the opening edge 21a of the sheet 2 to 50% of a distance between the opening edge 21a and the outer peripheral edge 22 (for example, a region of 20 to 50%). The fixing filamentary body 33 is preferably sewn so as to cross over the sheet-side filamentary body 34 sewn to the sheet 2 (so as to overlap the sheet-side filamentary body 34). In this case, the fixing filamentary body 33 passes above the sheet-side filamentary body 34, so that the height of the protruded and recessed structure from the surface of the sheet 2 with the fixing filamentary body 33 and the sheet-side filamentary body 34 becomes high. Thus, the resistance of the fixing structure 1 moving relative to the wall-like tissue T becomes larger. Therefore, the sheet 2 and the wall-like tissue T are further suppressed from moving relative to each other in the extending direction of the sheet 2 parallel to the surface of the sheet 2. Moreover, in addition to the minute space SP between the fixing filamentary body 33 and the surface of the sheet 2 (see FIG. 5) and the minute space SP between the sheet-side filamentary body 34 and the surface of the sheet 2 (see FIG. 5), a minute space is also created between the fixing filamentary body 33 and the sheet-side filamentary body 34. Thus, the cell of the wall-like tissue T enters into these minute spaces, so that the healing of the incision site of the wall-like tissue Tis promoted, allowing the fixation structure 1 to be fixed early to the wall-like tissue T. Moreover, with the cell of the wall-like tissue T entering into these minute spaces, the fixing filamentary body 33 and the sheet-side filamentary body 34 become a constrained state by the entering cell. Thus, even if the entire fixation structure 1 is likely to move relative to the wall-like tissue T, the cell that has entered into the minute space becomes entangled with the fixing filamentary body 33 and the sheet-side filamentary body 34, suppressing the relative movement of the fixation structure 1 with respect to the wall-like tissue T.

In the present embodiment, as shown in FIGS. 16, 17, 21 and 23, the fixing structure 1 further comprises a sheet-side filamentary body 34 provided on the outside of the sandwiching part 5 and the sheet-side filamentary body 34 is sewn onto the surface of the sheet 2 so as to form a protruded and recessed structure providing resistance to a relative movement in the extending direction of the sheet 2 with respect to the target site. Similar to the filamentary body 3 of the first to fifth embodiments, the sheet-side filamentary body 34 forms a protruded and recessed structure providing resistance to a relative movement with respect to a target site. A part of the wall-like tissue T thereby enters into the recessed part of the protruded and recessed structure when the fixing structure 1 is provided on the target site (wall-like tissue T). Thus, the sheet 2 and the wall-like tissue T are suppressed from moving relative to each other in the extending direction of the sheet 2 parallel to the surface of the sheet 2. Thus, misalignment from the target site can be suppressed without sewing the sheet 2 to the target site such as the skin. The fixing structure 1 can be thereby fixed early to the target site by a simple procedure. As the sheet-side filamentary body 34, a filamentary body similar to the filamentary body 3 or the fixing filamentary body 33 described in the first to fifth embodiments can be used.

In the present embodiment, the sheet-side filamentary body 34 is a filamentary body sewn only to the sheet 2 and is not sewn to the sandwiching part 5 (the first sandwiching member 51 and the second sandwiching member 52). An extending direction of the sheet-side filamentary body 34 is not particularly limited as long as the sheet-side filamentary body 34 can form the above-mentioned protruded and recessed structure. For example, the sheet-side filamentary body 34 may extend in a radiating direction, may extend in a circumferential direction, or may extend inclining to the radiating direction or the circumferential direction. In the sheet 2, a region in which the sheet-side filamentary body 34 extends is not particularly limited as long as the sheet-side filamentary body 34 can form the above-mentioned protruded and recessed structure. For example, the sheet-side filamentary body 34 is preferably sewn so as to extend on the outside of the sandwiching part 5 and in a region from the opening edge 21a of the sheet 2 to 50% of a distance between the opening edge 21a and the outer peripheral edge 22 (for example, a region of 20 to 50%). Moreover, in the present embodiment, the sheet-side filamentary body 34 is shown as an intersecting sewing part 341 (see FIG. 23) that will be mentioned later. However, the sheet-side filamentary body 34 is not limited to the sewing pattern shown in FIG. 23, and may be a filamentary body 3 described in the first to fifth embodiments or may have another sewing pattern.

In the present embodiment, as shown in FIG. 23, the sheet-side filamentary body 34 has an intersecting sewing part 341 intersecting so as to overlap in the thickness direction of the sheet 2. The intersecting sewing part 341 is a sewing part having a structure in which at least two filamentary bodies extending in different directions intersect with each other. When the sheet-side filamentary body 34 has the intersecting sewing part 341, one of the filamentary bodies intersecting with each other overlaps with and rides on the other of the filamentary bodies. Thus, at an intersecting portion of the intersecting sewing part 341, a height of a protruded and recessed structure formed by the intersecting sewing part 341 becomes increased in a direction perpendicular to the surface of the sheet 2 (a thickness direction of the sheet 2). Thus, when the fixing structure 1 is arranged on a target site of a living body, a frictional force between the fixing structure 1 and the target site of the living body can be increased, and misalignment of the fixing structure 1 with respect to the target site such as the skin can be further suppressed. Moreover, minute spaces are created between filamentary bodies intersecting with each other at the intersecting sewing part 341. Thus, the fixing structure 1 forms a more complex three-dimensional structure, making it easier for cells to enter and settle.

In the present embodiment, as shown in FIG. 23, the intersecting sewing part 341 is formed by two filamentary bodies, that extend slightly inclining relative to a radiating direction from the inside to the outside of the sheet 2, intersecting with each other. However, as long as the intersecting sewing part 341 intersects so as to overlap in the thickness direction of the sheet 2, a direction in which the filamentary body of the intersecting sewing part 341 extends is not particularly limited. For example, the intersecting sewing part may be configured so that a filamentary body extending in a radial direction and a filamentary body extending in a circumferential direction intersect with each other in a cross shape. Moreover, the intersecting sewing part may be configured in which three or more filamentary bodies intersect with each other.

It should be noted that in FIG. 23, and FIGS. 24 to 28 relating to embodiments that will be mentioned later, the filamentary bodies (fixing filamentary body 33, sheet-side filamentary body 34) are shown with lines in a simplified manner, but in practice, the filamentary bodies have a predetermined thickness, and only a small clearance is formed between the through holes 23, 51a, 52a and the filamentary bodies, as described in the first embodiment (see the description regarding clearance CL in FIG. 6).

It should be noted that a method of manufacturing a fixing structure 1 in the present embodiment is not particularly limited, but by way of an example, the fixing structure 1 can be manufactured as follows. It should be noted that the following manufacturing method is merely an example, the manufacturing method of the fixing structure 1 is not limited, and the present invention is not limited to the following descriptions.

First, the sheet-side filamentary body 34 is sewn to the sheet 2 to form a sewn structure such as an intersecting sewing part 341 as shown in FIG. 21. With the first sandwiching member 51 arranged in the arrangement part 25 of the sheet 2 to which the sheet-side filamentary body 34 is sewn, the sheet 2 and the first sandwiching member 51 are arranged from one end side of the insertion part 4 to the mounting part 44 of the insertion part 4. Next, the second sandwiching member 52 is arranged on the other surface 2b side of the sheet 2 so as to be aligned along the opening edge 21a of the sheet 2. In this state, the second sandwiching member 52 is fastened to the fixing part 45 of the insertion part 4 by the fastening member SC. Next, the fixing filamentary body 33 is sewn to the sheet 2, the first sandwiching member 51, and the second sandwiching member 52 over the entire circumferential direction of the sheet 2. The sheet 2 is thereby stably fixed to the sandwiching part 5. Moreover, since the second sandwiching member 52 is fixed to the insertion part 4, the sheet 2, the sandwiching part 5, and the insertion part 4 are fixed to one another, and the sheet 2 is stably held against the insertion part 4. It should be noted that the fixing filamentary body 33 may be sewn to the sheet 2, the first sandwiching member 51, and the second sandwiching member 52 before the second sandwiching member 52 is fastened to the fixing part 45 of the insertion part 4.

### Seventh Embodiment

As shown in FIG. 24, the seventh embodiment differs from the sixth embodiment in that auxiliary sewing part 342 is provided, but configurations other than the provision of the auxiliary sewing part 342 are similar to those of the sixth embodiment. It should be noted that configurations described below can be applied to other embodiments, and configurations of other embodiments can be applied to this embodiment as well.

In this embodiment, as shown in FIG. 24, the sheet-side filamentary body 34 comprises, in addition to the intersecting sewing part 341, an auxiliary sewing part 342 provided adjacent to the intersecting sewing part 341 in the circumferential direction of the sheet 2, in a region between a central portion of the intersecting sewing part 341 (a predetermined region in a radial direction including an intersecting portion 341a) and an inner end 341b of the intersecting sewing part 341, in the direction connecting the opening edge 21a of the opening 21 and the outer peripheral edge 22 of the sheet 2.

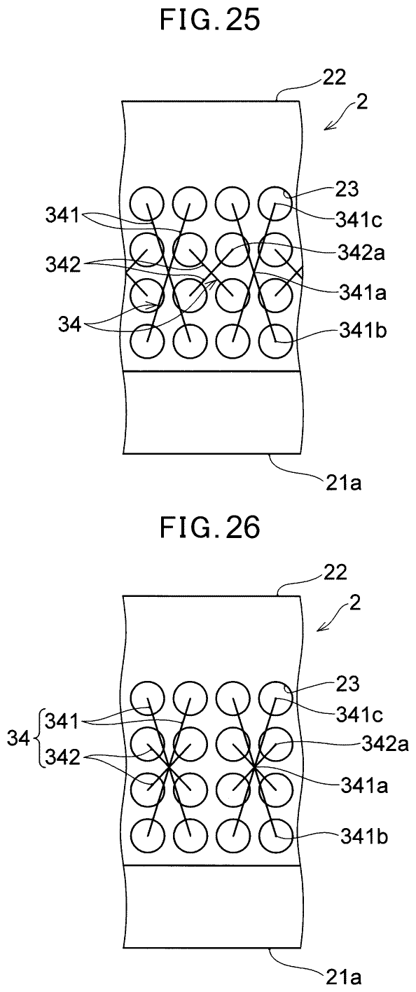

The auxiliary sewing part 342 is a sewing part auxiliary provided on the sheet 2 so as to reinforce the protruded and recessed structure formed by the intersecting sewing part 341. A structure of the auxiliary sewing part 342 is not particularly limited as long as it can reinforce the protruded and recessed structure formed by the intersecting sewing part 341. In this embodiment, the auxiliary sewing part 342 is shown as a linear sewing part extending radially. However, the auxiliary sewing part may extend in a circumferential direction, and may extend in a zigzag shape rather than in a linear shape. Moreover, as shown in FIG. 25, the auxiliary sewing parts 342 may intersect so as to overlap in the thickness direction of the sheet 2.

In this embodiment, as mentioned above, the auxiliary sewing part 342 is provided in the region between the central portion and the inner end 341b of the intersecting sewing part 341. The filamentary body can thereby become a denser state in the region between the central portion and the inner end 341b of the intersecting sewing part 341 by the intersecting sewing part 341 and the auxiliary sewing part 342. Thus, the rigidity of the sheet 2 becomes high in the region between the central portion and the inner end 341b of the intersecting sewing part 341. This makes the fixing structure 1 less likely to deform on the inner end 341b side of the intersecting sewing part 341. Thus, as mentioned above, the insertion member IS inserted into the fixing structure 1 can be maintained at a predetermined angle with respect to the target site (wall-like tissue T). Thus, the grown cell is suppressed from being peeled off between the fixing structure 1 and the wall-like tissue T due to the fixing structure 1 being deformed or tilted with respect to the wall-like tissue T. Therefore, the cell growth is not inhibited, and the cell proliferation on the inner end 341b side of the intersecting sewing part 341 becomes active, so that the healing of the incision site is promoted, and downgrowth can be suppressed. Besides, the inner end 341b of the intersecting sewing part 341 is provided at a position adjacent in a radial direction to the outer periphery of the sandwiching part 5 (first sandwiching member 51) on the surface (one surface 2a) of the sheet 2 (for example, in a range within 20% of a distance between the opening edge 21a and the outer peripheral edge 22 from the outer periphery of the first sandwiching member 51, although it is not limited to such a range). Moreover, the central portion of the intersecting sewing part 341 (intersecting portion 341a) is provided, but not limited to, for example, in a range within 30 to 50% of the distance between the opening edge 21a and the outer peripheral edge 22 from the outer periphery of the first sandwiching member 51.

Moreover, when the rigidity of the sheet 2 is increased in the region between the central portion and the inner end 341b of the intersecting sewing part 341, the sheet 2 becomes less likely to bend in a part of the sheet 2 adjacent in a radial direction to the outer periphery of the sandwiching part 5 (first sandwiching member 51) where the inner end 341b of the intersecting sewing part 341 is provided. Thus, even if a thin and flexible sheet 2 is used for a settlement part (flange-shaped part) of the fixing structure 1 onto the living body, it becomes possible to stably support the insertion part 4 and the insertion member IS each having predetermined size and weight at a predetermined angle. It should be noted that the description of “the region between the central portion and the inner end 341b of the intersecting sewing part 341” where the auxiliary sewing part 342 is provided refers to any region between the central portion and the inner end 341b. Thus, the auxiliary sewing part 342 may be provided only in a region corresponding to the central portion of the intersecting sewing part 341, but not in a region corresponding to the inner end 341b of the intersecting sewing part 341, may be provided only in the region corresponding to the inner end 341b, but not in the region corresponding to the central portion, or may be provided over the entire region corresponding from the central portion to the inner end 341b.

Moreover, in this embodiment, the outer end 342a of the auxiliary sewing part 342 is provided so as to be located inside the outer end 341c of the intersecting sewing part 341, as shown in FIGS. 24 and 25. The sheet-side filamentary body 34 provided on the sheet 2 thereby becomes sparser from the inside to the outside by the intersecting sewing part 341 and the auxiliary sewing part 342. Thus, as mentioned above, the sheet 2 is suppressed from suddenly changing in rigidity and being locally applied with stress, and the sheet 2 is suppressed from bending to cause the part to which the surface of the sheet 2 and the cell of the wall-like tissue T are fixed to peel off, suppressing delayed healing of the incision site.

### Eighth Embodiment

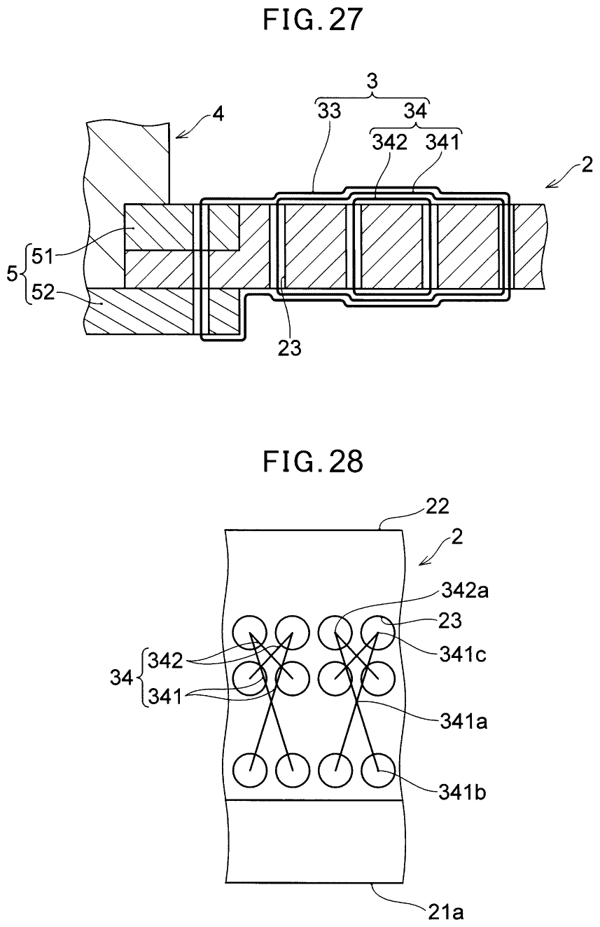

As shown in FIGS. 26 and 27, the eighth embodiment differs from the seventh embodiment in that auxiliary sewing part 342 is provided at a position overlapping with the intersecting sewing part 341, but configurations other than the provision of the auxiliary sewing part 342 are similar to those of the seventh embodiment. It should be noted that configurations described below can be applied to other embodiments, and configurations of other embodiments can be applied to this embodiment as well.

As shown in FIGS. 26 and 27, in this embodiment, as mentioned above, the auxiliary sewing part 342 is provided at a position overlapping with the intersecting sewing part 341 in the region between the central portion and the inner end 341b of the intersecting sewing part 341. When the intersecting sewing part 341 and the auxiliary sewing part 342 are provided so as to overlap with each other, a height in the thickness direction of the sheet 2 becomes high in a part where filamentary body of the intersecting sewing part 341 and filamentary body of the auxiliary sewing part 342 overlap with each other. Specifically, in addition to the intersection of the filamentary body of the intersecting sewing part 341 with each other, the filamentary body of the intersecting sewing part 341 intersect with the filamentary body of the auxiliary sewing part 342. Thus, the frictional force between the fixing structure 1 and the target site of the living body can be increased in the intersecting portion of the filamentary body of the intersecting sewing part 341 and the intersecting portion between the filamentary body of the intersecting sewing part 341 and the filamentary body of the auxiliary sewing part 342. Moreover, a minute space is generated also in a gap between the filamentary body of the intersecting sewing part 341 and the filamentary body of the auxiliary sewing part 342, forming a more complex three-dimensional structure and making it easier for cells to enter and settle. It should be noted that for the auxiliary sewing part 342, the phrase “provided at a position overlapping with the intersecting sewing part 341” means that a part of the filamentary body constituting the intersecting sewing part 341 and a part of the filamentary body constituting the auxiliary sewing part 342 are arranged overlapping so as to intersect with each other at any locations. Thus, the intersecting portion of the intersecting sewing part 341 and the intersecting portion of the auxiliary sewing part 342 may be misaligned. In this embodiment, as shown in FIG. 26, the intersecting portion of the intersecting sewing part 341 and the intersecting portion of the auxiliary sewing part 342 overlap in the thickness direction of the sheet 2. In this case, the height of the protruded and recessed structure becomes higher, so that the frictional force between the fixing structure 1 and the target site of the living body can be further increased. Moreover, the intersecting portion of the intersecting sewing part 341 and the intersecting portion of the auxiliary sewing part 342 form a more complex three-dimensional structure, making it easier for cells to enter and settle.

### Ninth Embodiment

As shown in FIG. 28, the ninth embodiment differs from the eighth embodiment in that a region where auxiliary sewing part 342 is provided is a region on an outer end 341c side of intersecting sewing part 341, but configurations other this configuration are similar to those of the eighth embodiment. It should be noted that configurations described below can be applied to other embodiments, and configurations of other embodiments can be applied to this embodiment as well.

In this embodiment, as shown in FIG. 28, an auxiliary sewing part 342 is provided at a position overlapping with intersecting sewing part 341 in a region on the outer end 341c side of the intersecting sewing part 341. When the auxiliary sewing part 342 is provided on the outer end 341c side of the intersecting sewing part 341, the number of regions having an intersecting structure increases, thereby making it easier for cells to enter and leading to an effect capable of suppressing misalignment. Furthermore, when the first sandwiching member 51 has a certain thickness, a difference in rigidity between the first sandwiching member 51 and the sheet 2 can be further reduced.

In FIG. 28, the auxiliary sewing part 342 is provided at a position overlapping with the intersecting sewing part 341, but it should be note that as a variation, the auxiliary sewing part may be provided adjacent to the intersecting sewing part 341 in the circumferential direction of the sheet 2 in the region on the outer end 341c side of the intersecting sewing part 341.

It should be noted that the embodiments mentioned above are merely examples, and the present invention is not limited to the embodiments mentioned above. For example, sewing patterns of filamentary body may be uniform in a circumferential direction, or may be switched to different sewing patterns in the circumferential direction. Moreover, positions of outer and/or inner ends of the filamentary body may vary in the circumferential direction.

Although the embodiments of the present invention have been described above, the present invention is not limited to the above-described embodiments. The above-described embodiments mainly explain the inventions having the following configurations.

(1) A fixing structure, comprising:
a porous sheet having an opening through which an insertion member led from inside of a living body to outside of the living body is insertable, wherein the sheet is composed of a non-resorbable material having biocompatibility;
a cylindrical insertion part having an insertion passage for guiding the insertion member from inside of the living body to outside of the living body;
a sandwiching part for sandwiching a peripheral portion around the opening of the sheet in a thickness direction of the sheet and being fixed to the insertion part; and
a fixing filamentary body composed of a filamentary body sewn to the sheet so as to fix the sheet and the sandwiching part,
wherein the fixing filamentary body is sewn to a surface of the sheet so as to form a protruded and recessed structure providing resistance to a relative movement in an extending direction of the sheet with respect to a target site of the living body where the insertion member is inserted.

(2) The fixing structure of (1), further comprising:
a sheet-side filamentary body provided on an outer side of the sandwiching part, wherein the sheet-side filamentary body is sewn onto the surface of the sheet so as to form a protruded and recessed structure providing resistance to a relative movement in the extending direction of the sheet with respect to the target site.

(3) The fixing structure of (1) or (2), wherein the sheet-side filamentary body has an intersecting sewing part intersecting so as to overlap in the thickness direction of the sheet.

(4) The fixing structure of any one of (1) to (3), wherein the sheet-side filamentary body comprises an auxiliary sewing part provided adjacent to the intersecting sewing part in the circumferential direction of the sheet or provided at a position overlapping with the intersecting sewing part, in a region between a central portion of the intersecting sewing part and an inner end of the intersecting sewing part, in a direction connecting an opening edge of the opening and an outer peripheral edge of the sheet.

(5) The fixing structure of any one of (1) to (4), wherein an outer end of the auxiliary sewing part is provided so as to be located inside an outer end of the intersecting sewing part.

(6) The fixing structure of any one of (1) to (5), wherein the auxiliary sewing part intersects so as to overlap in the thickness direction of the sheet.

(7) The fixing structure of any one of (1) to (6), wherein an intersecting portion of the intersecting sewing part and an intersecting portion of the auxiliary sewing part overlap in the thickness direction of the sheet.

(8) The fixing structure of any one of (1) to (7), wherein the sheet-side filamentary body comprises an auxiliary sewing part provided adjacent to the intersecting sewing part in a circumferential direction of the sheet or provided at a position overlapping with the intersecting sewing part, in a region on an outer end side of the intersecting sewing part, in a direction connecting an opening edge of the opening and an outer peripheral edge of the sheet.

(9) The fixing structure of any one of (1) to (8), wherein the auxiliary sewing part intersects so as to overlap in the thickness direction of the sheet.

(10) The fixing structure of any one of (1) to (9),
wherein the sandwiching part comprises a first sandwiching member that is annular and comes into contact with one surface of the sheet in the peripheral portion around the opening of the sheet and a second sandwiching member that is annular and comes into contact with an other surface of the sheet in the peripheral portion around the opening of the sheet,
wherein the first sandwiching member is composed of a hard biocompatible material having a predetermined rigidity higher than that of the sheet, and is arranged to surround a periphery of the insertion part, and
wherein the second sandwiching member is fixed to the insertion part.

(11) The fixing structure of any one of (1) to (10),
wherein the insertion part has a mounting part to which the peripheral portion around the opening of the sheet and an inner peripheral portion of the first sandwiching member are mounted,
wherein the mounting part has an engagement step configured to engage with a surface of the first sandwiching member opposite to a surface that comes into contact with the sheet in the thickness direction of the sheet, and
wherein the second sandwiching member is fastened to the insertion part so as to move in a direction approaching the first sandwiching member in the thickness direction of the sheet, so that the sheet and the first sandwiching member are compressed and sandwiched between the engagement step and the second sandwiching member.

(12) The fixing structure of any one of (1) to (11), wherein the sheet has an arrangement part for arranging the first sandwiching member on the peripheral portion around the opening, wherein the arrangement part is provided with a width corresponding to a width of the first sandwiching member in a direction connecting the opening edge of the opening and the outer peripheral edge of the sheet, and wherein a thickness of the sheet in the arrangement part is configured to be thinner than a thickness of the sheet outside the arrangement part.

(13) The fixing structure of any one of (1) to (12), wherein the sheet-side filamentary body is sewn to the sheet so that it changes from a denser state to a sparser state toward the outer peripheral edge side of the sheet from a central region side around the opening of the sheet.

(14) The fixing structure of any one of (1) to (13), wherein the filamentary body has a plurality of radial sewing parts extending in a radial direction connecting the opening and the outer peripheral edge of the sheet.

(15) The fixing structure of any one of (1) to (14), wherein the radial sewing part extends in a radial direction from the opening with a length that does not reach the outer peripheral edge.

(16) The fixing structure of any one of (1) to (15), wherein the radial sewing part has a plurality of first radial sewing parts each extending in a radial direction from the opening with a predetermined length and a plurality of second radial sewing parts each extending in a radial direction from the opening with a length shorter than that of the first radial sewing part.

### REFERENCE SIGNS LIST

1. Fixing structure
2. Sheet
2a. One surface of sheet
2b. Other surface of sheet
21. Opening of sheet
21a. Opening edge
22. Outer peripheral edge of sheet
23. Through hole
24. Non-sewing part
25. Arrangement part
3. Filamentary body
31. Radial sewing part
311. First radial sewing part
311a. Outer end of first radial sewing part
311b. Inner end of first radial sewing part
312. Second radial sewing part
312a. Outer end of second radial sewing part
312b. Inner end of second radial sewing part
313. Third radial sewing part
313a. Outer end of third radial sewing part
313b. Inner end of third radial sewing part
32. Circumferential sewing part
33. Fixing filamentary body
33a. Outer end of fixing filamentary body
33b. Inner end of fixing filamentary body
34. Sheet-side filamentary body
341. Intersecting sewing part
341a. Intersecting portion of intersecting sewing part
341b. Inner end of intersecting sewing part
341c. Outer end of intersecting sewing part
342. Auxiliary sewing part
342a. Outer end of auxiliary sewing part
4. Insertion part
4a. End face of insertion part
41. Insertion passage
42. First opening
43. Second opening
44. Mounting part
44a. Engagement step
45. Fixing part
5. Sandwiching part
51. First sandwiching member
51a. Through hole
52. Second sandwiching member
52a. Through hole
52b. Insertion part
C. Connecting member
C1. First connecting member
C2. Second connecting member
CL. Clearance between inner circumference of sheet and filamentary body
D. Fixing device
IS. Insertion member
Ra. Central region (First region)
Rb. Peripheral edge region (Fourth region)
Rc. Intermediate region
Rc1. First intermediate region (Second region)
Rc2. Second intermediate region (Third region)
SC. Fastening member
SP. Minute space
T. Wall-like tissue

1.-12. (canceled)

13. A fixing structure, comprising:
a porous sheet having an opening through which an insertion member led from inside of a living body to outside of the living body is insertable, wherein the sheet is composed of a non-resorbable material having biocompatibility;
a cylindrical insertion part having an insertion passage for guiding the insertion member from inside of the living body to outside of the living body;
a sandwiching part for sandwiching a peripheral portion around the opening of the sheet in a thickness direction of the sheet and being fixed to the insertion part; and
a fixing filamentary body composed of a filamentary body sewn to the sheet so as to fix the sheet and the sandwiching part,
wherein the fixing filamentary body is sewn to a surface of the sheet so as to form a protruded and recessed structure providing resistance to a relative movement in an extending direction of the sheet with respect to a target site of the living body where the insertion member is inserted.

14. The fixing structure of claim 13, further comprising:
a sheet-side filamentary body provided on an outer side of the sandwiching part,
wherein the sheet-side filamentary body is sewn onto the surface of the sheet so as to form a protruded and recessed structure providing resistance to a relative movement in the extending direction of the sheet with respect to the target site.

15. The fixing structure of claim 13, wherein the sheet-side filamentary body has an intersecting sewing part intersecting so as to overlap in the thickness direction of the sheet.

16. The fixing structure of claim 15, wherein the sheet-side filamentary body comprises an auxiliary sewing part provided adjacent to the intersecting sewing part in the circumferential direction of the sheet or provided at a position overlapping with the intersecting sewing part, in a region between a central portion of the intersecting sewing part and an inner end of the intersecting sewing part, in a direction connecting an opening edge of the opening and an outer peripheral edge of the sheet.

17. The fixing structure of claim 16, wherein an outer end of the auxiliary sewing part is provided so as to be located inside an outer end of the intersecting sewing part.

18. The fixing structure of claim 17, wherein the auxiliary sewing part intersects so as to overlap in the thickness direction of the sheet.

19. The fixing structure of claim 18, wherein an intersecting portion of the intersecting sewing part and an intersecting portion of the auxiliary sewing part overlap in the thickness direction of the sheet.

20. The fixing structure of claim 15, wherein the sheet-side filamentary body comprises an auxiliary sewing part provided adjacent to the intersecting sewing part in a circumferential direction of the sheet or provided at a position overlapping with the intersecting sewing part, in a region on an outer end side of the intersecting sewing part, in a direction connecting an opening edge of the opening and an outer peripheral edge of the sheet.

21. The fixing structure of claim 20, wherein the auxiliary sewing part intersects so as to overlap in the thickness direction of the sheet.

22. The fixing structure of claim 13,
wherein the sandwiching part comprises a first sandwiching member that is annular and comes into contact with one surface of the sheet in the peripheral portion around the opening of the sheet and a second sandwiching member that is annular and comes into contact with an other surface of the sheet in the peripheral portion around the opening of the sheet,
wherein the first sandwiching member is composed of a hard biocompatible material having a predetermined rigidity higher than that of the sheet, and is arranged to surround a periphery of the insertion part, and
wherein the second sandwiching member is fixed to the insertion part.

23. The fixing structure of claim 22,
wherein the insertion part has a mounting part to which the peripheral portion around the opening of the sheet and an inner peripheral portion of the first sandwiching member are mounted,
wherein the mounting part has an engagement step configured to engage with a surface of the first sandwiching member opposite to a surface that comes into contact with the sheet in the thickness direction of the sheet, and
wherein the second sandwiching member is fastened to the insertion part so as to move in a direction approaching the first sandwiching member in the thickness direction of the sheet, so that the sheet and the first sandwiching member are compressed and sandwiched between the engagement step and the second sandwiching member.

24. The fixing structure of claim 22, wherein the sheet has an arrangement part for arranging the first sandwiching member on the peripheral portion around the opening,
wherein the arrangement part is provided with a width corresponding to a width of the first sandwiching member in a direction connecting the opening edge of the opening and the outer peripheral edge of the sheet, and
wherein a thickness of the sheet in the arrangement part is configured to be thinner than a thickness of the sheet outside the arrangement part.
